(19) United States
(12) Patent Application Publication
(10) Pub. No.: US 2025/0279155 A1
(43) Pub. Date: Sep. 4, 2025

(54) CONSENSUS-BASED CLASSIFICATION TECHNIQUE TO DETERMINE GENETICALLY INFFERED ANCESTRY FROM COMPREHENSIVE GENOMIC PROFILING OF TUMOR DNA

(71) Applicant: OmniSeq, Inc., Buffalo, NY (US)

(72) Inventors: Zachary Wallen, Buffalo, NY (US); Sarabjot Pabla, Buffalo, NY (US); Eric Severson, Buffalo, NY (US); Mary K. Nesline, Buffalo, NY (US); Jeffrey Conroy, Buffalo, NY (US); Shakti Ramkissoon, Buffalo, NY (US)

(21) Appl. No.: 19/070,502

(22) Filed: Mar. 4, 2025

Related U.S. Application Data
(60) Provisional application No. 63/561,247, filed on Mar. 4, 2024.

(51) Int. Cl. G16B 10/00; G16B 20/20; G16B 20/40; G16B 40/30; G16B 50/30

(52) U.S. Cl. CPC G16B 10/00; G16B 20/20; G16B 20/40; G16B 40/30; G16B 50/30

(57) ABSTRACT

The disclosure relates to comprehensive genomic profiling (CGP) and to consensus-based classification techniques for determining genetically inferred ancestry from CGP of tumor DNA. Aspects are directed towards accessing reference and subject sequencing files and identifying genomic variants using a hybrid variant tool. The reference variant file is consolidated into a datastore formatted file that is queried to perform joint variant calling to generate a final reference variant file. The final reference variant file and the subject variant file are merged. On the merged variant file, principal component (PC) analysis is performed, and the PCs are used by a first and second classification process to generate a first and second ancestry call. The merged variant file is input into a third classification process to generate a third ancestry call. A consensus genetically inferred ancestry (GIA) call is predicted based on the first, the second, and the third ancestry calls.

### CROSS-REFERENCE TO RELATED APPLICATION

The present application is a non-provisional application of and claims the benefit and priority under 35 U.S.C. 119(e) of U.S. Provisional Application No. 63/561,247, filed on Mar. 4, 2024, the entire contents of which is incorporated herein by reference in its entirety for all purposes.

### FIELD

The present disclosure is directed generally to comprehensive genomic profiling, more particularly to consensus-based classification techniques for determining genetically inferred ancestry from comprehensive genomic profiling of tumor DNA.

### BACKGROUND

Comprehensive genomic profiling (CGP) represents a transformative next-generation sequencing (NGS) approach that uses a single assay to detect and characterize genomic alterations across hundreds of genes. These alterations include small DNA variants such as single nucleotide variants (SNVs) and small insertions or deletions (indels), as well as copy number variations (CNVs), gene fusions, splice variants, tumor mutational burden, and microsatellite instability. The ability to identify such a wide range of genomic signatures makes CGP an invaluable tool in diverse applications, including cancer diagnostics, organ transplant monitoring, and non-invasive prenatal testing (NIPT). By analyzing either tissue samples or liquid biopsies—such as tumor tissue, donor and recipient blood samples, or maternal blood—CGP provides clinicians with detailed insights into a patient's genomic landscape, enabling precise and informed decision-making regarding personalized healthcare strategies.

Next-generation sequencing (NGS), also referred to as massively parallel sequencing, enables the concurrent sequencing of millions of DNA bases, revolutionizing genomic testing. NGS begins with clonal amplification, a process in which DNA fragments from a patient sample are amplified and bound to a flow cell. Sequencing by synthesis is then performed, where fluorescently labeled nucleotides compete for addition to a growing DNA strand based on the template sequence. A light source excites the fluorescence signal unique to each nucleotide, and the emission wavelength and signal intensity are analyzed to determine the base call. With the ability to sequence hundreds to millions of DNA templates within each flow cell lane, NGS offers unparalleled flexibility and sensitivity in genetic screenings. These high-throughput platforms support targeted sequencing of single genes, multi-gene panels, whole exomes, and even whole genomes, significantly enhancing the accuracy and scalability of genomic testing.

The choice of NGS assay depends on the specific clinical or research objective. Single-gene panels, for instance, focus on a specific gene, such as BRCA1, to detect variants or alterations within the targeted region. However, single-gene panels often fail to cover the entire gene sequence, increasing the risk of missing clinically significant alterations outside the targeted region. Moreover, performing multiple single-gene tests can rapidly deplete sample specimens, necessitating additional biopsies—a costly and invasive process for patients. Multi-gene panels provide broader coverage by targeting multiple genes or regions associated with a particular disease or diagnostic purpose. However, similar limitations exist, as these panels may overlook genomic alterations outside the targeted scope. Comprehensive panels, which include hundreds of genes, offer the most extensive coverage. For example, comprehensive panels may focus on cancer-related genes, newborn genetic screening, or transplant-related genes. Although whole-genome sequencing (WGS) provides the most complete genomic landscape, its clinical adoption remains limited due to high costs, complex datasets, and inconsistent insurance coverage.

Recent advancements in CGP have expanded its utility beyond traditional diagnostics to address disparities in clinical research involving understudied minority populations. Historically, studies examining cancer progression, severity, and treatment responsiveness have relied on self-identified race and ethnicity (SIRE) data. However, SIRE data lacks consistency across studies, relies on broad racial or ethnic categories, and often fails to accurately reflect a patient's genetic background. To address these shortcomings, genetic ancestry inferred from common single nucleotide polymorphisms (SNPs) has emerged as a valuable alternative to complement or replace SIRE data. Using CGP and NGS methods such as targeted sequencing, whole-exome sequencing (WES), and WGS, researchers can integrate genetic ancestry into clinical studies to better understand population-specific differences in disease and treatment outcomes. This approach underscores the importance of genomic diversity in advancing precision medicine and improving healthcare equity.

### SUMMARY

Consensus-based classification techniques are disclosed herein (e.g., a computer implemented method, system and operations thereof, and non-transitory computer-readable medium storing code or instructions executable by one or more processors) for determining genetically inferred ancestry from comprehensive genomic profiling of tumor DNA.

In various embodiments, a computer-implemented method is provided comprising: accessing reference sequencing files and a subject sequencing file, wherein the reference sequencing files and the subject sequencing file are generated as part of performing a next generation sequencing assay; identifying, using a hybrid variant tool, genomic variants in the reference sequencing files and the subject sequencing file to generate reference variant files and a subject variant file; generating, by file consolidation using the reference variant files, a datastore formatted file; performing, by querying the datastore formatted file, joint variant calling to aggregate the variant calls across the reference variant files to generate a final reference variant file; merging the final reference variant file with the subject variant file to generate a merged variant file; determining, by principal component (PC) analysis on the merged variant file, reference PCs and subject PCs, wherein the reference PCs and the subject PCs comprise a top set of PCs; predicting, by a first classification process using the reference PCs and the subject PCs, a first ancestry call based on the correlations found between the reference PCs and subject PCs; determining, by a second classification process using the reference PCs and the subject PCs, a second ancestry call based on a distance metric of the second classification process, and determining, by a third classification process using the merged variant file, a third ancestry call based on a maximum likelihood estimation of the third classification process; and predicting, for the subject sample, a consensus genetically inferred ancestry (GIA) call based on the first ancestry call, the second ancestry call, and the third ancestry call.

In some embodiments, the reference sequencing files comprise individual sequencing files from at least 6 ancestral populations, and wherein the ancestral populations comprise African, European, Admixed American, East Asian, South Asian, Middle Eastern, Central Asian/Siberian, Oceania populations, or any combination thereof.

In some embodiments, the subject sequencing file comprises gene regions corresponding to a comprehensive genome panel.

In some embodiments, the hybrid variant tool comprises a variant caller integrated into a genomic data analyzer.

In some embodiments, the variant caller uses multi-threading and distributed computing techniques, and wherein the genomic data analyzer uses FPGA processing.

In some embodiments, the top set of PCs comprises at least 20 principal components.

In some embodiments, the first classification method is a correlation-based algorithm that calculates the Pearson correlation between genetic PCs of the subject sample and PCs of every reference sample, extracts the top 1% of calculated correlations, and wherein the first ancestry call is the reference population with the highest number of reference samples represented in the top correlations.

In some embodiments, the second classification method is a k-nearest neighbor algorithm trained on the top set of PCs to predict the second ancestry call, and wherein the second ancestry call is the reference population that appears the most frequently in the k nearest neighbors.

In some embodiments, third classification method is an admixture method, and wherein the third ancestry call is the reference population with the highest ancestry fraction.

In some embodiments, the predicted consensus GIA call is reported as: (i) an ancestry type when at least two of either the first, the second, or the third ancestry calls are the same, (ii) mixed ancestry when all the maximum likelihood estimations from the third classification process are below a threshold, or (iii) inconclusive when no concordance across the first, second, and third ancestry calls and all the maximum likelihood estimations from the third classification process are below a threshold.

In some embodiments, a system is provided that includes one or more processors, and one or more computer-readable media storing instructions which, when executed by the one or more processors, cause the system to perform operations in any of the computer implemented methods disclosed herein.

In some embodiments, a computer-program product is provided that is tangibly embodied in a non-transitory computer-readable memory that includes instructions which, when executed by one or more processors, cause the one or more processors to perform operations in any of the computer implemented methods disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 shows a Table with characteristics of the current and previous workflows for estimating genetic ancestry from CGP sequencing results. Superscript letters beside study names denote studies that used the same or similar workflows. A=Foundation Medicine workflow, B=Dana-Farber Cancer Institute and Harvard University workflow, C=Memorial Sloan Kettering (MSK) Cancer Center workflow. Cells with “X” represent ancestries that were attempted to be inferred for patients. Cells with “-” indicate characteristics that did not apply to the study. Numbers preceded by a “˜” are approximated numbers due to reporting of numbers not being exact or having to be derived from inexact sources within the manuscript such as a table or figure. NR=not reported in the study's publication and could not be inferred based on referencing a previously published workflow. *=percentage derived from a figure. k-NN=k Nearest Neighbor. AFR, African; AMR, Admixed American; CAS/SIB, Central Asian/Siberian; EAS, East Asian; EUR, European; OCN, Oceania; SAS, South Asian

FIG. 2 shows a computing environment in accordance with various embodiments.

FIG. 3 shows a workflow for estimating the genetically inferred ancestry (GIA) of a patient in accordance with various embodiments.

FIG. 4 shows a flowchart illustrating a process for predicting the genetically inferred ancestry of a patient in accordance with various embodiments.

FIG. 5 shows a map of geographical populations included as reference samples in the GIA workflow.

FIG. 6 shows reference sample processing and data generation. White boxes indicate files that have been previously generated or are produced as part of the workflow. Numbers correspond to the main steps of the reference sample processing: (1) CRAM to BAM file conversion, (2) DRAGENSTR model construction, (3) sample-level variant calling with GATK-DRAGEN, (4) sample-level variant call consolidation, (5) joint variant calling, and (6) post variant call processing. Gray arrows or boxes represent processes that occur during the workflow. White boxes with bold borders correspond to inputs for variant calling. Abbreviations: STR, short tandem repeat; TSO, TruSight® Oncology; SNP, single-nucleotide polymorphism; GVCE, genomic variant call format; PCA, principal component analysis; AFR, African; AMR, Admixed American; CAS SIB, Central Asian Siberian; EAS, East Asian; EUR, European; MEA, Middle Eastern; OCN, Oceania; SAS, South Asian.

FIG. 7 shows an exemplary GIA workflow for inferring patient ancestry from TSO 500 sequencing results. White boxes indicate files that have been previously generated or are produced as part of the workflow. Numbers correspond to the main steps of the GIA workflow: (1) DRAGENSTR model construction, (2) sample-level variant calling with GATK-DRAGEN, (3) post variant call processing and merging with reference data set variants, and (4) GIA calling and consensus determination. Gray arrows and boxes represent processes that occur during the workflow. White boxes with bold borders correspond to inputs for variant calling. Of note, admixture analysis is only performed for seven (excluding the Oceania population) due to a bias noted during testing. Abbreviations: STR, short tandem repeat; SNP, single nucleotide polymorphism; VCE, variant call format; MAF, minor allele frequency; HWE, Hardy-Weinberg Equilibrium; PC, principal component; k-NN, k-Nearest Neighbor; AFR, African; AMR, Admixed American; CAS SIB, Central Asian Siberian; EAS, East Asian; EUR, European; MEA, Middle Eastern; SAS, South Asian.

FIGS. 8A-G show representative plots for each ancestry group showing examples of results derived from the principal component (PC) correlation-based algorithm. The top 20 genetic PCs of each patient derived from PCA with reference samples were correlated with the top 20 genetic PCs of each reference sample using Pearson correlation. Plots of PC1 and 2 were then annotated with the correlation coefficients to determine how well the correlation strengths were capturing the patient's relationship to other reference samples in the PCA. Plots for the FIG. 8A African, FIG. 8B Admixed American, FIG. 8C East Asian, FIG. 8D European, FIG. 8E Middle Eastern, FIG. 8F South Asian, and FIG. 8G Central Asian/Siberian ancestry groups are shown here for representative patients of each group. Each point represents a unique reference sample (N=3,592) or patient tumor sample. Points colored by correlation coefficient correspond to reference samples, while black points correspond to the representative patient. No consensus GIA calls relating to Oceania ancestry were made during technical validation of the GIA workflow, therefore, representative plots for these populations are not shown.

FIG. 9 shows the process for determining a consensus genetically inferred ancestry (GIA) for a patient based on GIA calls from the three individual classification methods (k-NN, PC correlation, admixture analysis). Dark blue boxes correspond to conditions in the consensus determination process. Light blue or pink boxes correspond to a consensus GIA output that results from a certain condition being met.

FIGS. 10A-D show results from performing GIA workflow for 504 patients whose tumors were tested via CGP as part of their standard care. FIG. 10A shows a projection of patient data points who had non-inconclusive consensus GIA (N=501) onto reference samples (N=3592) within a combined patient PCA plot. Each point represents a unique reference sample (circles) or patient (diamonds). Points are colored by reference sample ancestral populations or if patients were determined to be of mixed ancestry (dark diamond points). Distances between points represent how similar genetic PCs were between a reference sample or patient compared to others in the plot. A full breakdown of GIA calls for patients including inconclusive patients is provided as a table within the PCA plot. FIG. 10B shows the relationship between patients' SIRE and their consensus GIA call shown via Sankey chart (N=491 patients with SIRE data). The size of nodes on each side of the chart and links in the middle represent the number of patients belonging to a group (nodes) or the number of patients of a certain race or ethnicity being classified into an ancestral group (links). For plot clarity, patient numbers were log-transformed before plotting. FIG. 10C shows ancestral fractions for patients with non-inconclusive consensus GIA, calculated via ADMIXTURE. Each bar represents what fraction of ancestry is contributed by each ancestral population for a unique patient, adding up to 1 (100%). FIG. 10D shows distributions of ancestral fractions from ADMIXTURE within each GIA group. The bottom, middle, and top horizontal boundaries of each box in the box plots represent the first, second (median), and third quartiles of the data for a particular GIA group. The lines extending from the two ends of each box represent 1.5× outside the interquartile range. Points beyond the lines are considered outliers. Abbreviations: AFR, African; AMR, Admixed American; CAS SIB, Central Asian Siberian; EAS, East Asian; EUR, European; MEA, Middle Eastern; OCN, Oceania; SAS, South Asian.

FIG. 11 is a Table showing the self-reported race and ethnicity and genetically inferred ancestry (GIA) of patients who had testing performed twice on same or different tumor specimens. Shades of grey for rows indicates results belonging to the same patient. Cells with “NA” denote missing data. Cells with “-” denote patient or tissue data that remained consistent between first and second testing. NOS, not otherwise specified.

FIG. 12 shows distributions of mapped sequences of patients within each GIA group. Differences in mapped sequences between consensus GIA groups were assessed using Wilcoxon rank-sum test on log transformed mapped sequence counts and uncorrected P-values from testing are shown above each bar that denotes the pairwise comparisons. The bottom, middle, and top horizontal boundaries of each box in the box plots represent the first, second (median), and third quartiles of the data for a particular GIA group. The lines extending from the two ends of each box represent 1.5× outside the interquartile range. Points beyond the lines are considered outliers. Abbreviations: AFR, African; AMR, Admixed American; EAS, East Asian; EUR, European; MEA, Middle Eastern; SAS, South Asian.

FIG. 13 shows differences in overall ancestry fractions between White patients with no reported ethnicity and White patients reporting their ethnicity as Native American. Each column represents the mean of each group and error bars extending from columns represents the standard error of the mean. Differences between groups was tested using Wilcoxon rank-sum test and resulting P-values are shown above each plot in parentheses. Abbreviations: AFR, African; AMR, Admixed American; CAS SIB, Central Asian Siberian; EAS, East Asian; EUR, European; MEA, Middle Eastern; SAS, South Asian; P, P-value from Wilcoxon rank-sum test.

FIGS. 14A and 14B show classification performance of GIA workflow compared to patients' SIRE. Classification performance for GIA classification methods was measured to assess how well GIA calls recapitulated SIRE of patients. FIG. 14A shows confusion matrices showing the relationship between SIRE (observed) vs GIA (predicted) of each patient for k-Nearest Neighbor (k-NN) (top row), PC correlation (second row), ADMIXTURE (third row), and consensus GIA (fourth row). Observed columns correspond to given 1000 Genomes ancestry populations based on patients' self-identified race. Predicted rows correspond to GIA calls derived from the classification algorithms. Green boxes indicate concordance between observed and predicted classifications aligned. Concordance percentages were calculated by summing the values in green boxes and dividing by the total number of patients with SIRE data for each ancestry group and for the total cohort (bold text). Mixed ancestry predictions were not applicable to k-NN and PC correlation methods, therefore, those rows do not have values. Classifications that were only available with GIA calls are highlighted light grey. FIG. 14B shows classification performance metrics calculated from confusion matrices for individual classification methods and consensus GIA. Performance metrics were calculated for each ancestral population independently, then averaged across populations to get overall performance metrics (column “Mean±SD”). For all metrics, the higher the value, the better a method performed based on that metric. Performance metrics were not calculated for “Middle Eastern” or “Mixed Ancestry” groups as no self-identified race corresponding to those groups were recorded for patients. Abbreviations: AFR, African; AMR, Admixed American; EAS, East Asian; EUR, European; MEA, Middle Eastern; SAS, South Asian; SD, standard deviation.

FIGS. 15A-D show concordance of consensus GIA classifications compared to a patients' SIRE by tumor type and tumor characteristics. FIG. 15A shows concordances of consensus GIA compared to SIRE for each tumor type. For each tumor type, concordance percentages were calculated by summing the number of times GIA matched with a patient's SIRE and dividing by the total number of patients with SIRE data for each tumor type. FIG. 15B shows the proportion of GIA calls within each tumor type. FIG. 15C shows concordances of consensus GIA compared to SIRE for each tumor type after excluding ancestry groups that were not covered by SIRE (MEA, Mixed Ancestry, Inconclusive). FIG. 15D shows distributions of matches and mismatches between consensus GIA calls and SIRE among different levels of tumor mutational burden (TMB), microsatellite instability (MSI), and different tissue specimen sites from where tumor biopsies were taken (primary vs metastatic tissue sites). Differences were tested for using Fisher exact tests and values above bars represent the uncorrected P-values. Abbreviations: AFR, African; AMR, Admixed American; EAS, East Asian; EUR, European; MEA, Middle Eastern; SAS, South Asian.

### TERMS

As used herein, the terms “about,” “similarly,” “substantially,” and “approximately” and are defined as being largely but not necessarily wholly what is specified (and include wholly what is specified) as understood by one of ordinary skill in the art. In any disclosed embodiment, the term “about,” “similarly,” “substantially,” or “approximately” may be substituted with “within [a percentage] of” what is specified, where the percentage includes 0.1 percent, 1 percent, 5 percent, and 10 percent, etc.

As used herein, when an action is “based on” something, this means the action can be based at least in part on at least a part of the something.

As used herein, “biomarker” refers to an observable indicator, such as a predictive, diagnostic, and/or prognostic sign, that can be identified in a sample. The biomarker functions as a signal for a specific subtype of a disease or disorder (e.g., cancer), characterized by particular molecular, pathological, histological, and/or clinical features. In certain instances, a biomarker may manifest as a gene. Biomarkers encompass a variety of entities, including but not limited to polynucleotides (e.g., DNA and/or RNA), alterations in polynucleotide copy numbers (e.g., DNA copy numbers), polypeptides, modifications to polypeptides and polynucleotides (e.g., posttranslational modifications), carbohydrates, and/or glycolipid-based molecular markers.

As used herein, “cancer” refers to an abnormal state or condition characterized by rapidly proliferating cell growth. Rapidly proliferating cells may be categorized as pathologic (i.e., characterizing or constituting a disease state), or may be categorized as non-pathologic (i.e., a deviation from normal but not associated with a disease state). In general, a cancer will be associated with the presence of one or more tumors (i.e., abnormal cell masses). The term “tumor” is meant to include all types of cancerous growths or oncogenic processes, metastatic tissues or malignantly transformed cells, tissues, or organs, irrespective of histopathologic type or stage of invasiveness. Examples of cancer include malignancies of various organ systems, such as lung cancers, breast cancers, thyroid cancers, lymphoid cancers, gastrointestinal cancers, and genito-urinary tract cancers. Cancer can also refer to adenocarcinomas, which include malignancies such as colon cancers, renal-cell carcinoma, prostate cancer and/or testicular tumors, non-small cell carcinoma of the lung, cancer of the small intestine, and cancer of the esophagus. Carcinomas are malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. An “adenocarcinoma” refers to a carcinoma derived from glandular tissue or in which the tumor cells form recognizable glandular structures. A “sarcoma” refers to a malignant tumor of mesenchymal derivation. “Melanoma” refers to a tumor arising from a melanocyte. Melanomas occur most commonly in the skin and are frequently observed to metastasize widely.

As used herein, “nucleic acid” or “nucleotide” refers to deoxyribonucleotides or ribonucleotides and polymers thereof in either single- or double-stranded form and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acid as used herein also refers to nucleic acids that have the same basic chemical structure as a naturally occurring nucleic acid. Such analogues have modified sugars and/or modified ring substituents but retain the same basic chemical structure as the naturally occurring nucleic acid. A nucleic acid mimetic refers to chemical compounds that have a structure that is different the general chemical structure of a nucleic acid, but that functions in a manner similar to a naturally occurring nucleic acid. Examples of such analogues include, without limitation, phosphorothioates, phosphoramidates, methyl phosphonates, chiral-methyl phosphonates, 2-O-methyl ribonucleotides, and peptide-nucleic acids (PNAs). The term “oligonucleotide” refers to a relatively short polynucleotide (e.g., less than about 250 nucleotides in length), including, without limitation, single-stranded deoxyribonucleotides, single- or double-stranded ribonucleotides, RNA: DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

As used herein, “optional” or “optionally” means that the subsequently described event or circumstance can or cannot occur, and that the description includes instances where the event or circumstance occurs and instances where it does not. For example, the phrase optionally the composition can comprise a combination means that the composition may comprise a combination of different molecules or may not include a combination such that the description includes both the combination and the absence of the combination (i.e., individual members of the combination).

As used herein, “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

### DETAILED DESCRIPTION

### Introduction

Clinical research has historically relied on participants from a single self-identified race to mitigate effects of underlying population differences. This has led to large gaps in the understanding of how cancer impacts diverse populations and reduces the generalizability of clinical research findings. Knowledge gaps and the limitations of research generalizability hinders cancer prevention efforts and optimization of patient treatment strategies for minority groups, leading to racial disparities in cancer outcomes. For example, only 4% of Blacks participated in Oncology trials in 2023. Black and American Indian and Alaskan Native men have the highest overall cancer mortality rates, 18% higher than White men, and Black women have 40% higher breast cancer death rates than White women. Patients from diverse communities' face barriers at the individual, interpersonal, institutional and policy level that limit participation in clinical research. Additionally, clinical studies seeking to understand patient differences based on genetic ancestry have relied on SIRE as a proxy measure. However, SIRE is not consistently assessed using a universal standard, is often derived from questions with a small number of broad racial or ethnic categories that may or may not relate to a given patient, and concordance between SIRE and genetic ancestry can vary. These challenges, among others, have impeded advancements of including diverse populations in clinical research.

In place of SIRE, recent cancer studies have incorporated genetic ancestry information inferred from common SNPs in a patient's genome detected using different sequencing modalities including whole-genome, whole-exome, genotyping, targeted gene panels, and RNA sequencing. Overall, genetic ancestry provides a more precise and objective assessment of an individual's “biogeographical” ancestry compared to SIRE. However, accurate ancestry inference from tumor-derived DNA sequences is challenging due to the presence of somatic mutations, loss of heterozygosity, microsatellite instabilities, and other genomic abnormalities which can disrupt calling of germline SNPs. Moreover, the targeted gene panels that typify CGP assays in standard clinical use present additional challenges as they only measure a fraction of the genome, are enriched in genes prone to somatic mutations compared to other genomic regions, and target coding regions of genes where ancestry-informative variants are sparse. While sequencing or whole genome genotyping of normal tissue can avoid some issues with tumor DNA, this testing is not yet feasible in the real-world patient healthcare setting which is usually restricted to patient tissue and liquid biopsies taken during standard care.

Despite the challenges, clinical studies have attempted to infer ancestry from CGP of patient samples such as tumor samples using a variety of workflows. These workflows typically involve (i) assignment of discrete ancestries through principal component analysis (PCA) of patient SNPs followed by classification with a machine learning algorithm and/or (ii) calculation of ancestral admixtures of patients providing quantitative measurements of ancestry. Both approaches involve training algorithms on genetic data from large, diverse reference populations, usually the 1000 Genomes (1000G) dataset. However, there remain areas to be improved upon within these workflows. Two key areas to be improved upon include the reference dataset used for ancestry inference and the algorithms used for determining consensus. Previous workflows performed ancestry inferences using five (or less) of the main 1000G populations (African, European, Admixed American, East Asian, South Asian), which do not fully capture all the potential ancestries a patient might have. These workflows have also relied on separate use of one or two types of algorithms to make ancestry inferences, which opens their ancestry inferences to method choice bias, reduces the ability to detect “inconclusive” ancestry inferences, and potentially increases the likelihood of false positives.

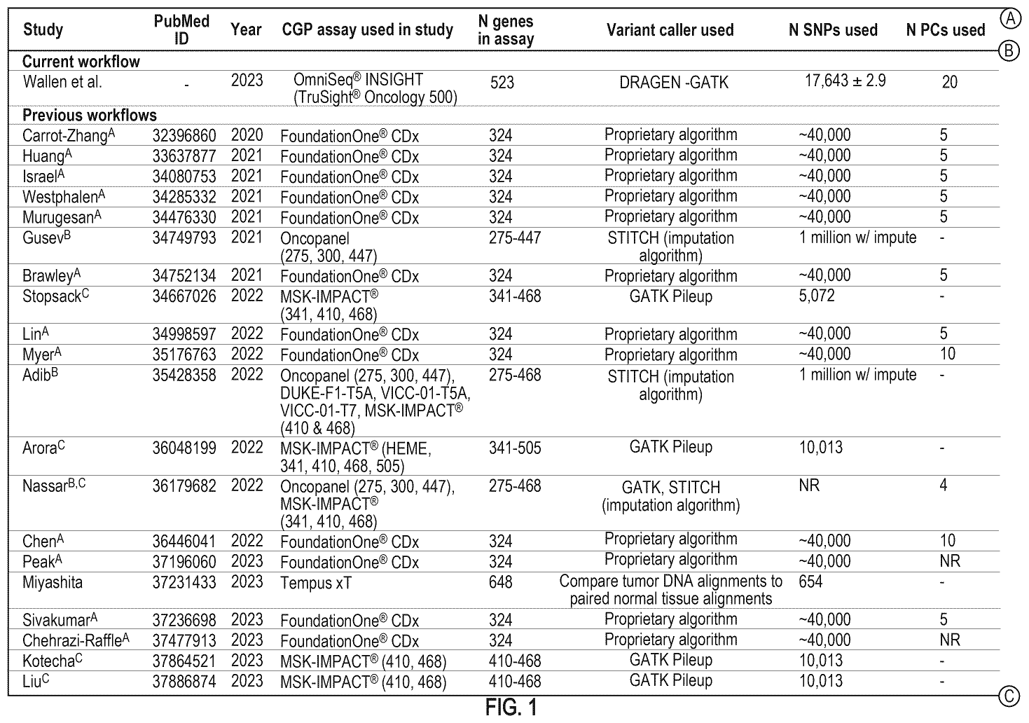

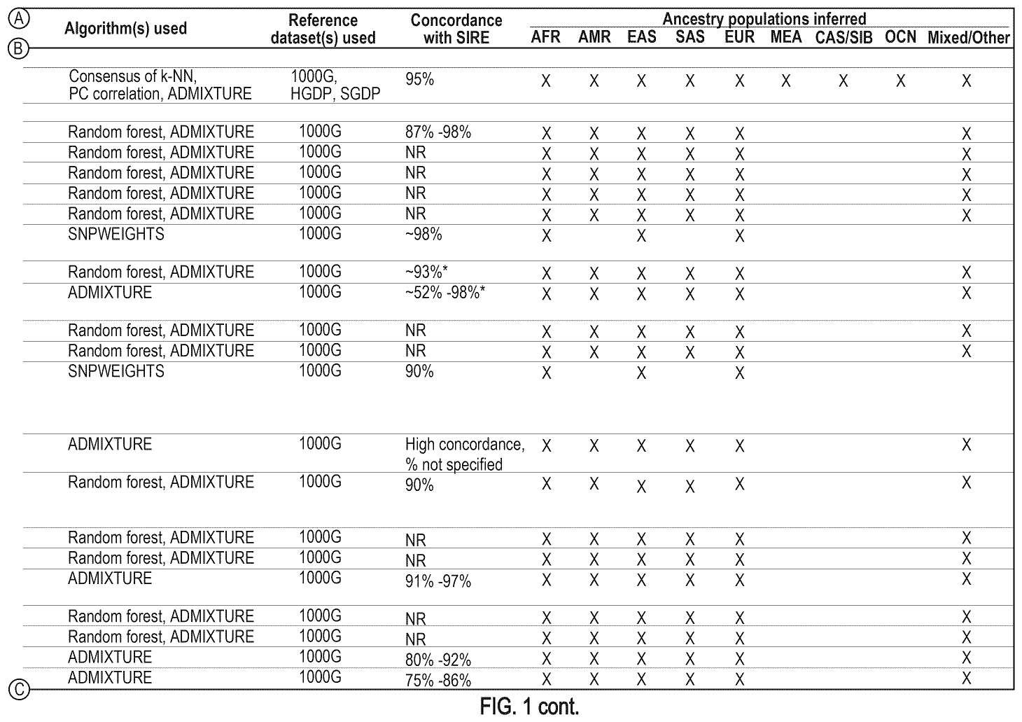

To overcome the above-mentioned challenges and other technical challenges discussed herein, the workflow described herein enhances these two main components that conventional workflows fall short in: the reference dataset used as an input for ancestry inference and the ancestry inference process or method itself (as described in further detail below and illustrated in FIG. 1).

### Increased Diversity in Reference Datasets

The CGP workflow described herein introduces significant advancements in genetic ancestry inference by expanding the diversity of reference datasets. While conventional workflows rely on the main 1000 Genomes populations—typically covering five or fewer population groups—the present workflow integrates additional populations, for example, additional populations including Middle Eastern, Central Asian/Siberian, and Oceania ancestries. This enhancement increases the diversity of reference populations from fewer than five to at least eight distinct groups, thereby significantly broadening the geographical coverage of the reference dataset. The inclusion of these underrepresented populations addresses critical gaps in conventional workflows, which often misclassify individuals due to the absence of appropriate reference populations. For instance, individuals with Middle Eastern ancestry are frequently misclassified as European or South Asian in conventional workflows, leading to inaccuracies in genetic ancestry inference and downstream analyses. By incorporating these additional populations, the present workflow ensures more accurate and representative classifications for individuals across a wider range of ancestries.

### Technological Challenges in Expanding Reference Populations

Expanding the diversity of reference populations is not without technological challenges. As the number of populations increases, so does the genetic correlation between them, creating difficulties in distinguishing closely related ancestry groups. To address this issue, the present workflow doubles the number of genetic principal components (PCs) used for ancestry inference, increasing the resolution from 10 PCs in conventional workflows to 20 PCs. This enhancement enables more precise differentiation of discrete ancestry groups by capturing finer variations in genetic data. Additionally, assembling diverse reference datasets requires robust population sampling strategies and the inclusion of high-quality genomic data from underrepresented groups, which can be logistically and ethically challenging. Nonetheless, these efforts are important to achieving the improved diversity and accuracy of ancestry inference described herein.

### Improvements in Classification Algorithms

The present workflow also introduces significant advancements in the algorithms used for genetic ancestry inference. Conventional workflows typically rely on one or two algorithms, such as random forest for estimating discrete ancestry populations and ADMIXTURE or SNPWEIGHTS for ancestral fractions. These approaches often analyze results separately, introducing method choice bias and limiting the robustness of ancestry estimations. In contrast, the described workflow employs multiple complementary algorithms to derive consensus-based ancestry inferences. Specifically, the workflow integrates two principal component-based classification methods—k-nearest neighbors (K-NN) and principal component (PC) correlation—alongside ADMIXTURE analysis to generate a unified consensus.

The use of K-NN and PC correlation in conjunction with ADMIXTURE provides for an improvement over conventional workflows that rely on random forest and/or ADMIXTURE alone. K-NN leverages the spatial distribution of individuals within the principal component space, enabling precise classification of individuals based on proximity to reference population clusters. PC correlation further refines this process by quantifying the relationship between individual genetic profiles and reference population PCs, ensuring robust ancestry assignments even in cases of overlapping genetic signals. ADMIXTURE complements these methods by estimating ancestral fractions and genetic admixture, providing quantitative insights into the proportion of ancestry derived from multiple populations.

The integration of these algorithms into a consensus-based framework reduces bias introduced by individual method choices and allows for inconclusive calls when robust inferences cannot be made, enhancing the reliability of ancestry estimations. Furthermore, the combination of K-NN and PC correlation is unique because it requires the simultaneous application of spatial and statistical methodologies, which are not traditionally paired, especially in ancestry inference workflows. By contrast, random forest algorithms are less effective in capturing the nuanced relationships within high-dimensional PC spaces, and ADMIXTURE alone lacks the capacity to classify discrete ancestry populations with high precision. The consensus framework described herein achieves a concordance rate of 95% with self-identified race and ethnicity (SIRE) data, matching or exceeding the performance of conventional workflows while offering greater methodological rigor.

### Agnostic Application to CGP Assays

Although the workflow is exemplified herein using oncology assays such as the TruSight® Oncology 500 (TSO 500) CGP assay, it is designed to be assay-agnostic and can be easily adapted to other CGP platforms without departing from the scope of the disclosure. For cancer applications, compatible CGP assays include FoundationOne® CDx, MSK-IMPACT (Memorial Sloan Kettering-Integrated Mutation Profiling of Actionable Cancer Targets), Tempus xT CDx, Guardant360, NeoGenomics Solid Tumor NGS Fusion Panel, GEM ExTra, and TissueNext. These assays analyze large numbers of genes across various genomic alterations, including tumor-specific mutations, copy number variations, and gene fusions. Beyond cancer, the workflow can be applied to non-cancer CGP assays, such as transplant gene panels like AlloSure® and Prospera™, which monitor donor-derived cell-free DNA for graft health and rejection, and non-invasive prenatal testing (NIPT) assays like Panorama®, Harmony®, and MaterniT21™, which assess fetal DNA for chromosomal abnormalities and genetic disorders. The versatility of the workflow underscores its broad applicability across diverse clinical and research contexts, further enhancing its utility in advancing precision medicine.

In one exemplary embodiment, a computer-implemented method is provided comprising: accessing reference sequencing files and a subject sequencing file, wherein the reference sequencing files and the subject sequencing file are generated as part of performing a next generation sequencing assay; identifying, using a hybrid variant tool, genomic variants in the reference sequencing files and the subject sequencing file to generate reference variant files and a subject variant file; generating, by file consolidation using the reference variant files, a datastore formatted file; performing, by querying the datastore formatted file, joint variant calling to aggregate the variant calls across the reference variant files to generate a final reference variant file; merging the final reference variant file with the subject variant file to generate a merged variant file; determining, by principal component (PC) analysis on the merged variant file, reference PCs and subject PCs, wherein the reference PCs and the subject PCs comprise a top set of PCs; predicting, by a first classification process using the reference PCs and the subject PCs, a first ancestry call based on the correlations found between the reference PCs and subject PCs; determining, by a second classification process using the reference PCs and the subject PCs, a second ancestry call based on a distance metric of the second classification process, and determining, by a third classification process using the merged variant file, a third ancestry call based on a maximum likelihood estimation of the third classification process; and predicting, for the subject sample, a consensus genetically inferred ancestry (GIA) call based on the first ancestry call, the second ancestry call, and the third ancestry call.

### Computing Environment

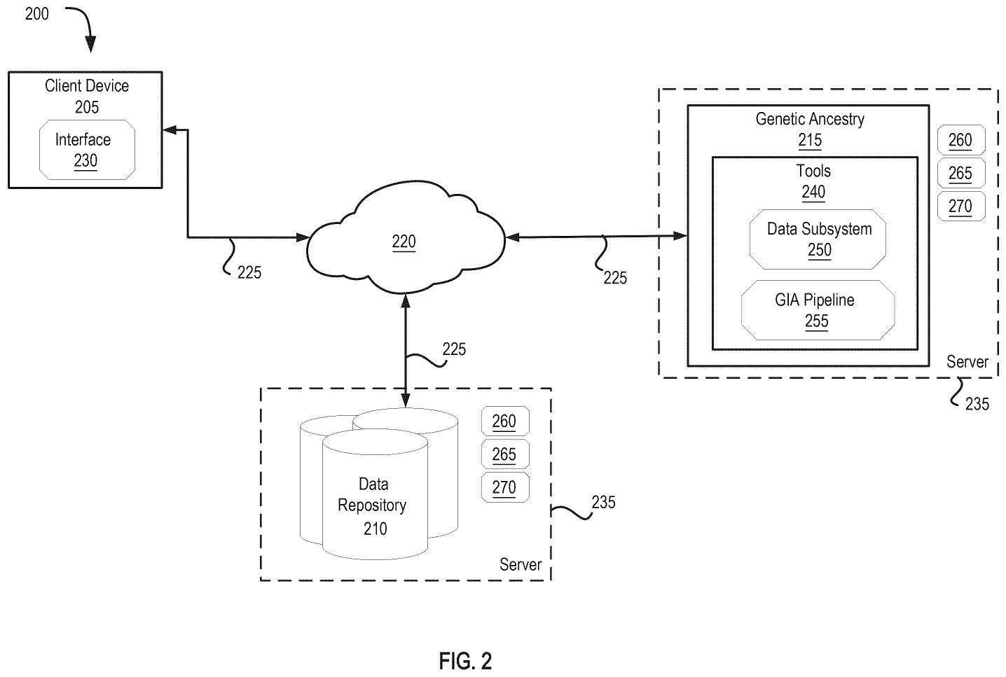

FIG. 2 shows a computing environment 200 in accordance with aspects of the present disclosure. Computing environment 200 includes a client device 205, a data repository 210, and a genetic ancestry platform 215 connected to each other by a network 220. Although FIG. 2 illustrates a particular arrangement of a client device 205, a data repository 210, genetic ancestry platform 215, and a network 220, this disclosure contemplates any suitable arrangement of a client device 205, a data repository 210, genetic ancestry platform 215, and a network 220. As an example, and not by way of limitation, two or more client devices 205, a data repository 210, and genetic ancestry platform 215 may be connected to each other directly, bypassing network 220. As another example, two or more client devices 205, a data repository 210, and a genetic ancestry platform 215 may be physically or logically co-located with each other in whole or in part. Moreover, although FIG. 2 illustrates a particular number of a client device 205, a data repository 210, a genetic ancestry platform 215, and network 220, this disclosure contemplates any suitable number of client devices 205, data repositories 210, genetic ancestry platform 215, and networks 220. As an example, and not by way of limitation, computing environment 200 may include multiple client devices 205, data repositories 210, genetic ancestry platform 215, and networks 220.

This disclosure contemplates any type of network 220 familiar to those skilled in the art that may support data communications using any of a variety of available protocols including without limitation TCP/IP (transmission control protocol/Internet protocol), SNA (systems network architecture), IPX (Internet packet exchange), AppleTalk®, and the like. Merely by way of example, network(s) 220 may be a local area network (LAN), networks based on Ethernet, Token-Ring, a wide-area network (WAN), the Internet, a virtual network, a virtual private network (VPN), an intranet, an extranet, a public switched telephone network (PSTN), an infra-red network, a wireless network (e.g., a network operating under any of the Institute of Electrical and Electronics (IEEE) 1002.11 suite of protocols, Bluetooth®, and/or any other wireless protocol), and/or any combination of these and/or other networks.

Links 225 may connect a client device 205, a data repository 210, and a genetic ancestry platform 215 to a network 220 or to each other. This disclosure contemplates any suitable links 225. In particular embodiments, one or more links 225 include one or more wireline (such as for example Digital Subscriber Line (DSL) or Data Over Cable Service Interface Specification (DOCSIS)), wireless (such as for example Wi-Fi or Worldwide Interoperability for Microwave Access (WiMAX)), or optical (such as for example Synchronous Optical Network (SONET) or Synchronous Digital Hierarchy (SDH)) links. In particular embodiments, one or more links 225 each include an ad hoc network, an intranet, an extranet, a VPN, a LAN, a WLAN, a WAN, a WWAN, a MAN, a portion of the Internet, a portion of the PSTN, a cellular technology-based network, a satellite communications technology-based network, another link 225, or a combination of two or more such links 225. Links 225 need not necessarily be the same throughout a computing environment 200. One or more first links 225 may differ in one or more respects from one or more second links 225.

A client device 205 is an electronic device including hardware, software, or embedded logic components or a combination of two or more such components and is capable of interacting with the data repository 210 and the genetic ancestry platform 215 with respect to appropriate product target discovery functionalities in accordance with techniques of the disclosure. The client devices may include various types of computing systems such as portable handheld devices, general purpose computers such as personal computers and laptops, workstation computers, wearable devices, gaming systems, thin clients, various messaging devices, sensors or other sensing devices, and the like. These computing devices may run various types and versions of software applications and operating systems (e.g., Microsoft Windows®, Apple Macintosh®, UNIX® or UNIX-like operating systems, Linux or Linux-like operating systems such as Google Chrome™ OS) including various mobile operating systems (e.g., Microsoft Windows Mobile®, iOS®, Windows Phone®, Android™, BlackBerry®, Palm OS®). Portable handheld devices may include cellular phones, smartphones, (e.g., an iPhone), tablets (e.g., iPad®), personal digital assistants (PDAs), and the like. Wearable devices may include Google Glass® head mounted display, and other devices. The client device 205 may be capable of executing various applications such as various Internet-related apps, communication applications (e.g., E-mail applications, short message service (SMS) applications) and may use various communication protocols. This disclosure contemplates any suitable client device 205 configured to generate and output product target discovery content to a user. For example, users may use client device 205 to execute one or more applications, which may generate one or more discovery or storage requests that may then be serviced in accordance with the teachings of this disclosure. A client device 205 may provide an interface 230 (e.g., a graphical user interface) that enables a user of the client device 205 to interact with the client device 205. The client device 205 may also output information to the user via this interface 230. Although FIG. 2 depicts only one client device 205, any number of client devices 205 may be supported.

A data repository 210 is a data storage entity (or sometimes entities) into which data has been specifically partitioned for an analytical or reporting purpose. The data repository 210 may be used to store data and other information for use by the genetic ancestry platform 215 and client device 205. For example, one or more of the data repositories 210(a) and 210(b) may be used to store data and information to be used as input into the genetic ancestry platform 215 for generating an estimated genetically inferred ancestry call for a patient. In some instances, the data and information relate to raw or processed genetic sequences (genomic, exome, and/or targeted), variant call format (VCF) files, files processed and output by the genetic ancestry platform 215, and other information used by the genetic ancestry platform 215 when performing assay functions. The data repositories 210 may reside in a variety of locations including servers 235. For example, a data repository used by server 235 may be local to server 235 or may be remote from server 235 and in communication with server 235 via a network-based or dedicated connection of network 220. Data repositories 210(a) and 210(b) may be of different types or of the same type. In certain examples, a data repository may be a database which is an organized collection of data stored and accessed electronically from one or more storage devices such as one or more servers 235. The one or more servers 235 may be configured to execute a database application that provides database services to other computer programs or to computing devices (e.g., client device 205 and genetic ancestry platform 215) within the computing environment, as defined by a client-server model.

The genetic ancestry platform 215 comprises a set of tools 240 for the purpose of analyzing and visualizing data (i.e., data stored in data repository 210). The genetic ancestry platform 215 is used to execute a process to estimate the GIA of a patient and report out their GIA calls. In the configuration depicted in FIG. 2, the set of tools 240 include two subsystems: a data subsystem 250 and a GIA subsystem 255. The subsystems are predefined operating environments through which the system or platform coordinates the workflow and resource used. Additionally, the subsystems can be run on the same hardware such as a processor or different hardware. The data subsystem 250 is responsible for loading, processing, and saving data accessed from the data repository 210 to be used by the data subsystem 250 itself or the GIA processor 255. The GIA subsystem 255 uses the processed data to estimate the GIA of a patient. In some instances, the genetic ancestry platform 215 detects and processes genomic variants, generates genetic principal components (PCs), and inputs genetic PCs into (i) a k-nearest neighbor (kNN) algorithm and (ii) a correlation-based algorithm that calculates the Pearson correlation between genetic PCs of the patient and the genetic PC of ancestry population groups. In other instances, the genetic ancestry platform 215 may also perform admixture analysis to predict the ancestry fractions of a patient being contributed by each ancestry population group. The genetic ancestry platform 215 uses outputs from all three aforementioned methods to derive a consensus GIA call. The genetic ancestry platform 215 may reside in a variety of locations including servers 235. For example, a genetic ancestry platform 215 used by server 235 may be local to server 235 or may be remote from server 235 and in communication with server 235 via a network-based or dedicated connection of network 220. The genetic ancestry platform 215 may be of different configurations or of the same configuration. The one or more servers 235 may be configured to execute a discovery application that provides discovery services to other computer programs or to computing devices (e.g., client device 205) within the computing environment, as defined by a client-server model.

In various instances, server 235 may be adapted to run one or more services or software applications that enable one or more embodiments described in this disclosure. In certain instances, server 235 may also provide other services or software applications that may include non-virtual and virtual environments. In some examples, these services may be offered as web-based or cloud services, such as under a Software as a Service (SaaS) model to the users of client device 205. Users operating client device 205 may in turn utilize one or more client applications to interact with server 235 to utilize the services provided by these components (e.g., database and rescue applications). In the configuration depicted in FIG. 2, server 235 may include one or more components 260, 265 and 270 that implement the functions performed by server 235. These components may include software components that may be executed by one or more processors, hardware components, or combinations thereof. It should be appreciated that various device configurations are possible, which may be different from computing environment 200. The example shown in FIG. 2 is thus one example of a computing environment (e.g., a distributed system for implementing an example computing system) and is not intended to be limiting.

Server 235 may be composed of one or more general purpose computers, specialized server computers (including, by way of example, PC (personal computer) servers, UNIX® servers, mid-range servers, mainframe computers, rack-mounted servers, etc.), server farms, server clusters, or any other appropriate arrangement and/or combination. Server 235 may include one or more virtual machines running virtual operating systems, or other computing architectures involving virtualization such as one or more flexible pools of logical storage devices that may be virtualized to maintain virtual storage devices for the server. In various instances, server 235 may be adapted to run one or more services or software applications that provide the functionality described in the foregoing disclosure.

The computing systems in server 235 may run one or more operating systems including any of those discussed above, as well as any commercially available server operating system. Server 235 may also run any of a variety of additional server applications and/or mid-tier applications, including HTTP (hypertext transport protocol) servers, FTP (file transfer protocol) servers, CGI (common gateway interface) servers, JAVA® servers, database servers, and the like. Exemplary database servers include without limitation those commercially available from Oracle®, Microsoft®, Sybase®, IBM® (International Business Machines), and the like.

In some implementations, server 235 may include one or more applications to analyze and consolidate data feeds and/or data updates received from users of client computing devices 205. As an example, data feeds and/or data updates may include, but are not limited to, in vivo feeds, in silico feeds, or real-time updates received from public studies, user studies, one or more third party information sources, and data streams (continuous, batch, or periodic), which may include real-time events related to sensor data applications, biological system monitoring, and the like. Server 235 may also include one or more applications to display the data feeds, data updates, and/or real-time events via one or more display devices of client computing devices 205.

### Processing of Patient Samples

### Sample Acquisition

In many instances, a clinical diagnosis for a particular disease or condition (e.g., cancer, transplant rejection, genetic disease) or a treatment plan is made by genetically testing a sample collected from a patient at risk of/diagnosed with/being treated for a genetic disease. A sample can be a cell-containing liquid or a tissue comprising nucleic acid molecules. The sample can comprise, but is not limited to, amniotic fluid, tissue biopsies, blood, prenatal blood, blood cells, bone marrow, fine needle biopsy samples, peritoneal fluid, amniotic fluid, plasma, pleural fluid, saliva, semen, serum, tissue or tissue homogenates, frozen or paraffin sections of tissue. Methods of obtaining a sample include but are not limited to biofilms, aspirations, tissue sections, swabs, drawing blood or other fluids, surgical or needle biopsies, and the like. In various embodiments, the sample is a tumor sample obtained through tissue biopsy, liquid biopsy, and the like obtained from a patient at risk of/diagnosed with/being treated for one or more types of cancer.

As used herein, the terms “individual,” “patient,” and “subject” are used interchangeably. In certain embodiments, subjects are “patients,” i.e., living humans that are receiving medical care for a disease or condition. This includes persons with no defined illness who are being investigated for signs of pathology. Patients may have an existing tumor or have been diagnosed with a particular cancer. In some cases, a patient may suffer from one or more types of tumors/cancers simultaneously. For example, the subject can have pancreatic, kidney, renal, pelvic, colorectal, stomach, thymic, head and neck, mesothelial, prostate, cervical, thyroid, adrenal, testicular, breast, uterine, bone, esophageal, lung, liver, bile duct, ovarian, bladder, nervous system, and/or skin cancer. Furthermore, patients who are at risk of or have just been diagnosed with a disease (e.g., cancer) are likely to have not yet received cancer treatment(s). In other instances, a patient having been diagnosed with cancer is receiving cancer treatment(s).

To prepare patient samples for sequencing, nucleic acids (e.g., RNA and DNA) are isolated from the sample. In several embodiments, DNA is isolated from the sample(s). The DNA isolated/extracted from a sample may be whole genomic DNA, circulating cell-free DNA, ctDNA, mitochondrial DNA, circular DNA, tumor DNA, and the like. In some instances, the isolated DNA is tumor DNA. Various methods are known in the art for isolating DNA from a sample, such as using a reagent kit or by experimental means. A reagent kit (e.g., tubes and DNA extraction reagents, etc.) for library preparation may include materials such as probes for hybrid capture as well as any useful reagents & protocols for fragmentation, adapter ligation, purification/isolation, etc. Experimental methods for isolating/extracting DNA from a sample involve disruption and lysis of the starting material followed by the removal of proteins and other contaminants and finally recovery (e.g., alcohol precipitation) of the DNA.

In some instances, when it is determined that there is an insufficient amount of nucleic acid for analysis, amplification may be used to increase the amount of nucleic acid. Amplification refers to production of additional copies of a nucleic acid sequence and is generally carried out using polymerase chain reaction (PCR) or other technologies known in the art (e.g., Dieffenbach and Dveksler, PCR Primer, a Laboratory Manual, 1995, Cold Spring Harbor Press, Plainview, NY). PCR refers to methods by K. B. Mullis (U.S. Pat. Nos. 4,683,195 and 4,683,202, hereby incorporated by reference) for increasing concentration of a segment of a nucleic acid sequence in a mixture of genomic DNA without cloning or purification.

Following DNA isolation, the isolated DNA undergoes library preparation, which can include steps like fragmentation, end repair, adaptor ligation, enrichment, or any combination thereof. The isolated DNA are fragmented into a plurality of shorter double stranded DNA target fragments either physically (e.g., acoustic shearing, sonication) or enzymatically (e.g., with DNase I). Fragmentation, depending on the method, can generate DNA fragments ranging in size from 150 bp to 3 kb in length. Fragmentation can also cause short overhangs at 3′ and 5′ ends of the fragments. Any end repair methods known in the art may be used, such as an exonuclease reaction that adds an adenosine to overhangs. Ligation-based library preparations often make use of an adapter design which can incorporate an index sequence (e.g., a sample index sequence to identify sample origin for a nucleic acid sequence) via a ligase or polymerase to the end repaired DNA fragments. Adapter oligonucleotides are often complementary to flow-cell anchors and sometimes are utilized to immobilize a nucleic acid library to a solid support, such as the inside surface of a flow cell, for example. An adapter oligonucleotide may comprise an identifier, one or more sequencing primer hybridization sites (e.g., sequences complementary to universal sequencing primers, single end sequencing primers, paired end sequencing primers, multiplexed sequencing primers, and the like), or combinations thereof (e.g., adapter/sequencing, adapter/identifier, adapter/identifier/sequencing). Optionally, the DNA library, or parts thereof, are amplified (e.g., amplified by a PCR-based method). For example, a sequencing method may comprise amplification of a DNA library prior to or after immobilization on a bead or solid support (e.g., a solid support in a flow cell) using any suitable method. Library amplification is typically used as an enrichment step to increase the concentration of the library for sequencing.

### Sequencing Methods

The library prepped nucleic acids are sequenced using a machine capable of sequencing nucleic acids. Examples of sequencing may include, without limitation, NovaSeq, HiSeq, Genome Analyzer IIx, MiSeq, NextSeq, HiScanSQ, 454 DNA sequencer, GS FLX+, GS Junior System, OLID next-generation sequencing platform, Ion PGM System, Ion Proton System, Ion S5, Ion S5xl, CEQ 8000, RS system, Sequel system, nanopore sequencers, DNBSEQ-G50, DNBSEQ-G400, DNBSEQ-T7, Ultima Genomics UG100, etc. In certain instances, a full or substantially full sequence is obtained and sometimes a partial sequence is obtained.

Any suitable method of sequencing nucleic acids can be used, non-limiting examples of which include Maxim & Gilbert, chain-termination methods, sequencing by synthesis, sequencing by ligation, sequencing by mass spectrometry, microscopy-based techniques, the like or combinations thereof. In some embodiments, a first-generation technology, such as, for example, Sanger sequencing methods including automated Sanger sequencing methods, including microfluidic Sanger sequencing, can be used in a method provided herein. In some embodiments, sequencing technologies that include the use of nucleic acid imaging technologies (e.g., transmission electron microscopy (TEM) and atomic force microscopy (AFM)), can be used. In some embodiments, a high-throughput sequencing method is used. High-throughput sequencing methods generally involve clonally amplified DNA templates or single DNA molecules that are sequenced in a massively parallel fashion, sometimes within a flow cell. Next generation (e.g., 2^{nd} and 3^{rd} generation) sequencing techniques capable of sequencing DNA in a massively parallel fashion can be used for methods described herein and are collectively referred to herein as “massively parallel sequencing” (MPS). In certain embodiments, a non-targeted approach is used where most or all nucleic acids in a sample are sequenced, amplified and/or captured randomly.

Other suitable sequencing technologies may include single molecule, real-time (SMRT) technology of Pacific Biosciences (in SMRT, each of the four DNA bases is attached to one of four different fluorescent dyes. These dyes are phospholinked. A single DNA polymerase is immobilized with a single molecule of template single stranded DNA at the bottom of a zero-mode waveguide (ZMW) where the fluorescent label is excited and produces a fluorescent signal, and the fluorescent tag is cleaved off. Detection of the corresponding fluorescence of the dye indicates which base was incorporated); nanopore sequencing (DNA is passed through a nanopore and each base is determined by changes in current across the pore, as described in Soni & Meller, 2007, Progress toward ultrafast DNA sequence using solid-state nanopores, ClinChem 53(11):1996-2001); chemical-sensitive field effect transistor (chemPET) array sequencing (e.g., as described in U.S. Pub. 2009/0026082); and electron microscope sequencing (as described, for example, by Moudrianakis, E. N. and Beer M., in Base sequence determination in nucleic acids with the electron microscope, III. Chemistry and microscopy of guanine-labeled DNA, PNAS 53:564-71 (1965).

### First Generation Sequencing: Sanger

Sanger sequencing is a first-generation DNA sequencing method that has long been considered the gold standard for the accurate detection of single nucleotide variants (SNVs). First generation sequencing techniques, like Sanger, utilize a chain-termination method wherein specialized DNA bases (dideoxynucleotides or ddNTPs) are randomly incorporated into a growing DNA chain of nucleotides (A, C, G, T) generating different length DNA fragments. Fragments are size separated by capillary electrophoresis and a laser is used to excite the unique fluorescence signal associated with each ddNTP. As the fluorescence signal is recorded, a chromatograph is generated, showing which base is present at a given location of the target region being sequenced. In the clinical setting, Sanger provides flexibility for testing single or small batch samples (no more than a 10 gene panel) for prenatal, carrier, and genetic testing and can provide results in a relatively short period of time. However, Sanger is limited to short DNA sequences, approximately 300-1,000 bases, and can be more expensive compared to newer generation sequencing methods.

### Second Generation Sequencing: Next Generation Sequencing

NGS, can be applied in various sequencing techniques that include whole-genome sequencing (WGS), whole-exome sequencing (WES), targeted genome sequencing (TGS), and any other sequencing technique known to one skilled in the art may be performed on the prepared DNA library samples. NGS is based on the amplification of DNA on a solid surface using fold-back PCR and anchored primers. DNA is fragmented, and adapters are added to the 5′ and 3′ ends of the fragments. DNA fragments that are attached to the surface of flow cell channels are extended, bridge amplified, and denatured. Primers, DNA polymerase and four fluorophore-labeled, reversibly terminating nucleotides are used to perform sequential sequencing. After nucleotide incorporation, a laser is used to excite the fluorophores, an image is captured, and the identity of the first base is recorded. The 3′ terminators and fluorophores from each incorporated base are removed and the incorporation, detection and identification steps are repeated. Multiple cycles of the solid-phase amplification followed by denaturation can create several million clusters of approximately 1,000 copies of single-stranded DNA molecules of the same template in each channel of the flow cell. Sequencing according to this technology is described in U.S. Pat. Nos. 7,960,120; 7,835,871; 7,232,656; 7,598,035; 6,911,345; 6,833,246; 6,828,100; 6,306,597; 6,210,891; U.S. Pub. 2011/0009278; U.S. Pub. 2007/0114362; U.S. Pub. 2006/0292611; and U.S. Pub. 2006/0024681, each of which are incorporated by reference in their entirety.

Sequencing methods (e.g., NGS and Sanger) generate a large number of reads. As used herein, “reads” (e.g., “a read,” “a sequence read”) are short nucleotide sequences produced by any sequencing process described herein or known in the art. Reads can be generated from one end of nucleic acid fragments (“single-end reads”), and sometimes are generated from both ends of nucleic acid fragments (e.g., paired-end reads, double-end reads). The length of a sequence read is often associated with the particular sequencing technology. High-throughput methods, for example, provide sequence reads that can vary in size from tens to hundreds of base pairs (bp). Sequencing reads may have a mean, median, average, or absolute length of about 15 bp to about 1000 bp. For example, sequencing reads may be about 15 bp, 16 bp, 17 bp, 18 bp, 19 bp, 20 bp, 25 bp, 50 bp, 100 bp, 150 bp, 200 bp, 300 bp, 400 bp, 500 bp, 600 bp, 700 bp, 800 bp, 900 bp, or about 1000 bp or about any integer value between 15 bp and 1000 bp. Sequencing reads, and their associated quality scores, are stored in files known as FASTQ files or FASTA files. Typically, FASTQ files can comprise about 1 million to about 5 million reads per sample; however, more or less reads may be generated depending on the sample.

In some embodiments, sequence reads are generated, obtained, gathered, assembled, manipulated, transformed, processed, and/or provided by a sequence subsystem comprising a sequencer. A machine comprising a sequence subsystem can be a suitable machine and/or apparatus that determines the sequence of a nucleic acid utilizing a sequencing technology known in the art. In some embodiments a sequence subsystem can align, assemble, fragment, complement, reverse complement, and/or error check (e.g., error correct sequence reads). The sequence reads are processed using a sequence processing subsystem to obtain sequence read data. The processing of the sequence reads includes read alignment, mapping, and filtering. To perform all these processing steps, bioinformatics workflow can comprise any combination of bioinformatic techniques including demultiplexing, reference genome alignment, database design, and variant calling as nonlimiting examples.

As described above, the outputs of sequencing are FASTQ files that comprise all the reads for a single sample. Part of the process of generating FASTQ files is demultiplexing (e.g., sorting) all the different library samples that were pooled together in a single flow cell lane into their own FASTQ file. In a typical sequencing run, multiple library samples (e.g., 4, 12, 16, etc.) are combined and loaded onto a single lane of a sequencing flow cell. Because during the library preparation, each DNA fragment in a sample had a corresponding unique barcode ligated onto the fragments. Accordingly, when multiple libraries are pooled for sequencing, the barcodes allow for the samples to be distinguished from one another. The barcodes are also what are used to sort each sample into its own sequencing FASTQ file (i.e., demultiplexing).

Alignment of reads to a reference genome (e.g., a human reference genome) involves mapping any number of reads to a specified nucleic acid region (e.g., a chromosome or portion thereof) and are referred to as counts. As used herein, the term “reference genome” can refer to any known, sequenced or characterized genome, whether partial or complete, of any organism or virus which may be used to reference identified sequences from a subject. Suitable reference human genomes may include a published human genome (e.g., hg19 or hg38) or some other reference material, such as “gold standard” sequences obtained by, e.g., Sanger sequencing of subject nucleic acid.

Any suitable mapping/alignment method (e.g., process, algorithm, program, software, subsystem, the like or combination thereof) can be used. Non-limiting examples of computer algorithms that can be used to align sequences include, without limitation, BLAST, BLITZ, FASTA, BOWTIE 1, BOWTIE 2, ELAND, MAQ, PROBEMATCH, SOAP, BWA or SEQMAP, or variations thereof or combinations thereof. The terms “aligned,” “alignment,” or “aligning” generally refer to two or more nucleic acid sequences that can be identified as a match (e.g., 100% identity) or partial match. Alignments can be done manually or by a computer (e.g., a software, program, subsystem, or algorithm), non-limiting examples of which include the Efficient Local Alignment of Nucleotide Data (ELAND) computer program distributed as part of the Illumina Genomics Analysis pipeline. Alignment of a sequence read can be a 100% sequence match. In some cases, an alignment is less than a 100% sequence match (i.e., non-perfect match, partial match, partial alignment). In some embodiments an alignment is about a 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76% or 75% match. In some embodiments, an alignment comprises a mismatch. In some embodiments, an alignment comprises 1, 2, 3, 4 or 5 mismatches. Two or more sequences can be aligned using either strand (e.g., sense or antisense strand). In certain embodiments a nucleic acid sequence is aligned with the reverse complement of another nucleic acid sequence. The results from alignment are deposited in an alignment file (e.g., BAM).

As a quality control step, all alignment files may be filtered to remove non-primary alignment records, reads mapped to improper pairs, and reads with more than six edits. Individual bases are excluded if their Phred base quality is less than 30 in tumor samples and less than 20 in normal samples. As described herein, the term “less than” comprises all whole numbers and rational numbers. For example, less than 30 includes 29.9, 29.8, 29.7, 29.6, 29.5, 29.4, 29.3, 29.2, 29.1, 29.0, 25, 20, 15, 10, 5, and 0.

During reference genome alignment, variations between the sample and the reference genome may be identified. The process of comparing sequence data to a reference is called variant calling. As described herein, variants comprise naturally occurring alterations to a DNA sequence not found in the reference sequence, and the alterations can be classified as benign, likely benign, variant of unknown significance, likely pathogenic or pathogenic. Moreover, variants can comprise both germline variants (e.g., variants present in all the body's cells) and somatic variants (variants that arise during the lifetime of an individual, such as if an individual develops cancer). Examples of variants include small sequence variants (less than 50 base pairs) such as single nucleotide variants (SNVs), single nucleotide polymorphisms (SNPs) and small structural variants (SVs) (e.g., deletions, insertions, insertions and deletions, sometimes referred to as indels) and larger (greater than 50 base pairs) SVs such as chromosomal rearrangements (e.g., translocations and inversions) and copy number changes. SNVs/SNPs are the result of single point mutations that can cause synonymous changes (nucleotide change does not alter the encoded amino acid), missense changes (nucleotide change does alter the encoded amino acid), or nonsense changes (resulting amino acid change converts the encoded codon to a stop codon). Further, variants can occur in both coding and non-coding regions of the genome and can be detected by WGS, as opposed to targeted gene panels, and target specific probes.

Variant calling can use one or more variant calling tools to examine the aligned/mapped sequencing data and reference genome side-by-side to determine the existence of sequence mutations (single base changes and small indels). The variant calling tool may extract candidate variants from alignment data, score a number of individual metrics for each variant, and apply these scores both individually and in combination to identify bona fide sequence mutations and to exclude sequence artifacts. Any suitable technique/variant calling tool may be used to detect structural alterations such as, for example, MuTect, Strelka, dysgu, JointSNVMix2, GATK, DRAGEN, or any combination thereof. The list of detected variants and their properties (e.g., type of variant) are annotated and deposited in a variant file (e.g., variant call format (VCF)). The output VCF files can include about 1,500 to a few million variants; however, more or less variants may be found depending on the sample.

### Estimation of Genetically Inferred Ancestry Calls Using Comprehensive Genomic Profiling

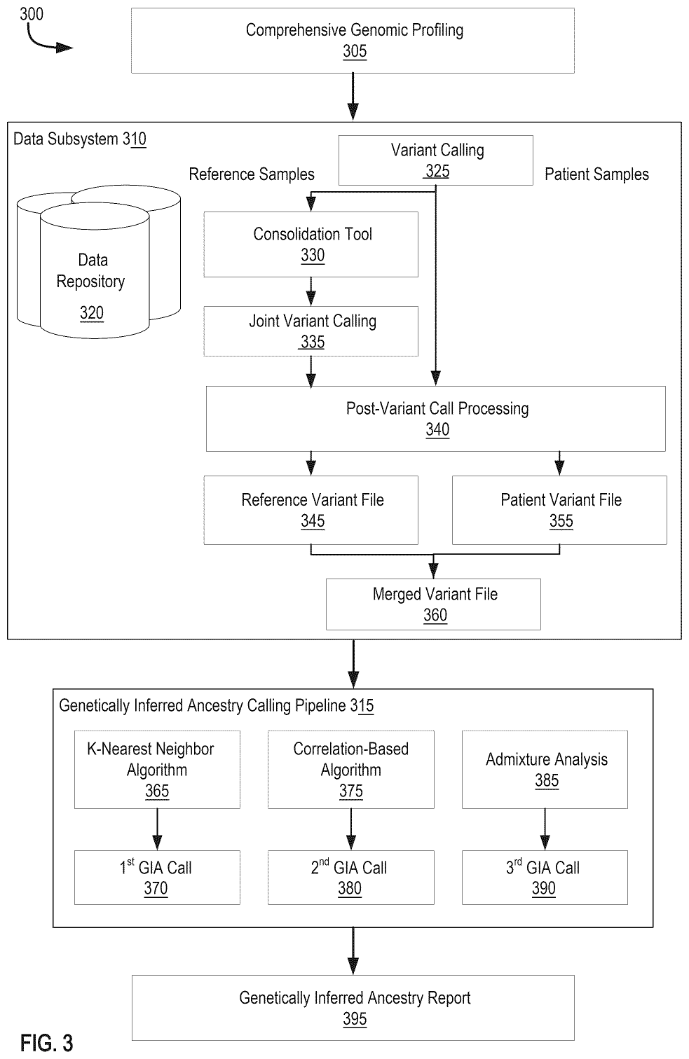

FIG. 3 shows a block diagram illustrating workflow 300 for estimating a genetically inferred ancestry report for a subject such as a patient. In some embodiments, processing steps shown in workflow 300 comprises performing a comparison (e.g., comparing a test profile to a reference profile). The test profile may be a patient variant file obtained from a comprehensive gene panel, while the reference profile may be a variant file that comprises variants identified from diverse set of reference populations. Two or more data sets, two or more relationships and/or two or more profiles can be compared by a suitable method. Non-limiting examples of statistical methods suitable for comparing data sets, relationships and/or profiles include Behrens-Fisher approach, bootstrapping, Fisher's method for combining independent tests of significance, Neyman-Pearson testing, confirmatory data analysis, exploratory data analysis, exact test, F-test, Z-test, T-test, calculating and/or comparing a measure of uncertainty, a null hypothesis, counter-nulls and the like, a chi-square test, omnibus test, calculating and/or comparing level of significance (e.g., statistical significance), a meta-analysis, a multivariate analysis, a regression, simple linear regression, robust linear regression, the like or combinations of the foregoing. In certain embodiments comparing two or more data sets, relationships and/or profiles comprises determining and/or comparing a measure of uncertainty. A “measure of uncertainty” as used herein refers to a measure of significance (e.g., statistical significance), a measure of error, a measure of variance, a measure of confidence, the like or a combination thereof. A measure of uncertainty can be a value (e.g., a threshold) or a range of values (e.g., an interval, a confidence interval, a Bayesian confidence interval, a threshold range). Non-limiting examples of a measure of uncertainty include p-values, a suitable measure of deviation (e.g., standard deviation, sigma, absolute deviation, mean absolute deviation, the like), a suitable measure of error (e.g., standard error, mean squared error, root mean squared error, the like), a suitable measure of variance, a suitable standard score (e.g., standard deviations, cumulative percentages, percentile equivalents, Z-scores, T-scores, R-scores, standard nine (stanine), percent in stanine, the like), the like or combinations thereof. In some embodiments determining the level of significance comprises determining a measure of uncertainty (e.g., a p-value). In certain embodiments, two or more data sets, relationships and/or profiles can be analyzed and/or compared by utilizing multiple (e.g., 2 or more) statistical methods (e.g., least squares regression, principal component analysis, Pearson correlation, admixture analysis, linear discriminant analysis, quadratic discriminant analysis, bagging, neural networks, support vector machine models, random forests, classification tree models, K-nearest neighbors, logistic regression and/or loss smoothing) and/or any suitable mathematical and/or statistical manipulations (e.g., referred to herein as manipulations).

Workflow 300 is implemented using a computing environment such as the computing environment 200 described with respect to FIG. 2. The goal of workflow 300 is to use SNP data from a large and diverse reference dataset with known ancestry and compare it to individual patient CGP results to obtain accurate genetically inferred ancestry information about the patient. By better understanding patient differences associated with genetic ancestry, clinicians and clinical researchers can better develop treatment plans tailored to each individual patient. For example, the race/ethnicity of a patient may determine which cancer biomarkers are expressed/present and help clinicians and doctors to diagnose and treat their patients more accurately. As way of example, without limitation, Ki-67 labeling of cell proliferation and invasiveness in breast cancer is higher in Arab/Moroccan patients compared to individuals with European ancestry. Another example includes biomarkers associated with the risk and clinical outcome of prostate cancer show differential expression between African Americans and European Americans.

To achieve this goal, various steps of workflow 300 are executed. Workflow 300 access and processes sequencing files (e.g., raw data, alignment files, variant files, and the like) for reference populations and patient CGPs from data repositories 320 (similar to data repository 210 with respect to FIG. 2). The reference sequencing files include thousands of individual sample files obtained from a variety of ancestral groups. Accordingly, the reference sequencing files have additional processing steps, such as file consolidation and joint variant calling, to aggregate the variants into a single variant file representing the genomic variants found across different ancestral backgrounds. On the other hand, variant calling is the only processing step performed on the patient sequencing file prior to merging the reference variant file with the patient variant file. The merged patient-reference variant file is then input into a genetically inferred ancestry calling pipeline for analysis by various algorithms to determine an estimated genetically inferred ancestry for the patient. A report is generated and made available for use in clinical research.

Initially, a patient sample is submitted for comprehensive genomic profiling (CGP) 305 to obtain sequencing read data for the identification of biomarkers associated with the patient's genetic ancestry. CGP 305 uses NGS assays (e.g., WGS, WES, TGS) to detect genomic variants common to a disease/condition and form gene panels. The gene panels range from large, broad coverage multi-gene panels containing hundreds of genes associated with a disease or test of interest. For example, comprehensive panels may target cancer-related genes on WGS, WES or tumor DNA. Alternatively, comprehensive gene panels may target genes related to NIPT or transplant screening. By including a large panel of genes associated with a disease, CGP poses several advantages compared to single-gene testing, namely that a large quantity of genes can be assessed at once without having to repeat tissue biopsy and laboratory processing. A full detailed description of acquiring a patient sample, processing the sample for sequencing, sequencing, and exemplary computations are provided above.

In some instances, CGPs are used to identify cancer biomarkers and genomic signatures (e.g., tumor mutational burden (TMB) and microsatellite instability (MSI)) to diagnose, treat, and/or predict patient survival. A cancer biomarker may be genomic alterations known to drive cancer growth. Genomic alterations (also referred to as genomic variants) are naturally occurring alterations to the DNA sequence not found in a reference sequence. Examples of genomic variants include small variants (less than 50 base pairs) such as single nucleotide variants (SNVs), single nucleotide polymorphisms (SNPs), insertions, and deletions (sometimes referred to as indels), and structural variants (greater than 50 base pairs) such as insertions, deletions, chromosomal rearrangements (e.g., translocations, inversions, and fusions), and copy number variations (CNVs). SNVs/SNPs are the result of single point mutations that can cause synonymous changes (nucleotide change does not alter the encoded amino acid), missense changes (nucleotide change does alter the encoded amino acid), or nonsense changes (resulting amino acid change converts the encoded codon to a stop codon). Common genomic alterations found in cancer include base substitutions, insertions, and deletions (also referred to as indels), copy number alterations/variants (CNA/Vs), and rearrangements or fusions of chromosomes.

TMB (as measured by DNA-sequencing methods known in the art) is a measure that quantifies the total number of genetic mutations (mutations per unit of DNA) within the coding region of a tumor's genome. These mutations can include single nucleotide substitutions, insertions, deletions, or other structural alterations. TMB is often expressed as the number of mutations per mega-base (Mb) of DNA. High TMB has been associated with better responses to immune checkpoint inhibitor therapies. More specifically, tumors with a high mutation burden are more likely to produce neoantigens (newly formed antigens) as a result of the mutations. These neoantigens can trigger an immune response. TMB may be assessed through next-generation sequencing (NGS) technologies, which allow for the comprehensive analysis of the tumor's genomic landscape.

Once the patient's sequencing data is obtained from the CGP 305, the data is input into data subsystem 310. Data subsystem 310 is responsible for performing the initial set of steps (e.g., loading data, preprocessing data, and saving data into data structures) to prepare the data (e.g., raw or processed sequencing data, variant files, and the like) for use in the genetically inferred ancestry calling pipeline 315. Data subsystem 310 is part of the framework for workflow 300 comprising hardware such as one or more processors (e.g., a CPU, GPU, TPU, FPGA, the like, or any combination thereof), memory, and storage that operates software or computer program instructions (e.g., Application Programming Interfaces (APIs), Cloud Infrastructure, Kubernetes, Docker, TensorFlow, Kuberflow, Torchserve, and the like) to execute arithmetic, logic, input and output commands in order to control the storage, organization, and retrieval of data. In some instances, data subsystem 310 implements deployment of the data using a cloud platform such as Amazon Web Services (AWS), Google Cloud Platform (GCP), and Microsoft Azure. As shown in FIG. 3, data subsystem 310 comprises data repositories 320 and a set of computational programs and tools for calling variants and preparing the variant files for input into the genetically inferred ancestry pipeline 315.

The data repositories 320 (e.g., data repository 210 as described with respect to FIG. 2) are configured as databases that store data obtained from publicly available data sources (e.g., 1000 Genomes (1000G) project data, the Human Genome Diversity Project (HGDP), Simons Genome Diversity Project (SGDP), and the like) and/or privately (e.g., in-house) processed data. The data obtained from publicly available data sources may comprise sequencing data files (e.g., raw files, alignment files, variant files, etc.) from individuals from various ancestral backgrounds. Examples may include, without limitation, African (AFR), Admixed American (AMR; mainly Native Central and South American ancestry), East Asian (EAS), European (EUR), and South Asian (SAS) populations. Additional ancestral populations accessed may further include Middle Eastern (MEA), Central Asian/Siberian (CAS/SIB), and Oceania (OCN) populations. In other instances, data repositories 320 can also store sequencing files of patient data obtained from CGP 305. The data stored in the data repositories 320 may be accessed by any of the sub-processors within data subsystem 310 for variant calling, variant processing, and the like. The types of data stored in data repositories 320 may include raw or processed sequencing data (e.g., patient comprehensive genome profiles 305 and publicly available datasets from various ancestral backgrounds), variant call files, processed variant call files, and any other information that may be necessary.

Sequencing data may include sequencing information related to the whole genome, exomes, tumor DNA, or targeted regions of interest (e.g., regions selected to appear in gene panels). In other instances, sequencing data may include sequencing files from a variety of ancestral groups obtained from public data sources. Data files (e.g., alignment files) obtained from public data sources may be in a different file format from one another and/or privately processed data. Accordingly, it may be necessary to convert data files from one file type to another. By way of example, publicly downloaded ancestral data for reference samples may be Compressed Reference oriented Alignment Map (CRAM) files, general feature format (GFF) files, BED files, SAM files, and the like, that need to be converted into a file type compatible with workflow 300 (e.g., binary alignment map (BAM)). File conversion tools for sequencing data that may be used include, without limitation, SAMtools, Picard tools, HTSlib, Seqtk, FASTX-Toolkit, BBTools, BAMTools, CRAMtools, bcftools, GATK, BEDtools, and the like.

As part of the preprocessing performed by data subsystem 310, the reference sequencing files may be filtered using the gene baits corresponding to the comprehensive gene panel used for the patient sample. The comprehensive gene panel may be any gene panel of interest such as cancer gene panels (e.g., TruSight Oncology 500® (TSO 500), FoundationOne®CDx, MSK-IMPACT, Tempus xT CDx, Guardant360, NeoGenomics Solid Tumor NGS Fusion Panel, GEM ExTra, and TissueNext), neonatal gene panels (e.g., Panorama®, Harmony®, and MaterniT21™), transplant gene panels (e.g., AlloSure® and Prospera™), and the like. Gene baits refer to custom-designed probes that are designed to target a predefined set of genes associated with a particular condition. After filtering with the gene panel, the reference sequencing files comprise targeted regions from the gene panel that are then processed by the sub-processing tools contained in data subsystem 310. In some instances, the gene panel is TSO 500 using the TSO 500 probes for filtering.

Depending on the sample type, e.g., reference versus patient, different processing steps are used by data subsystem 310. To identify genomic variants from the filtered reference samples sequencing files, the sub-processing tools for variant calling 325, sample-level variant calling consolidation tool 330, joint variant calling tool 335, and post-variant calling processing tool 340 are used on each reference sample independently. On the other hand, filtered patient sequencing files only use the variant calling 325 and post-variant calling processes. The genomic variants that can be identified in the filtered sequencing files include SNPs, indels, short tandem repeats (STRs), chromosomal rearrangements (e.g., translocations, inversions, and fusions), CNVs, and the like. Moreover, the genomic variants can comprise both germline variants (e.g., variants present in all the body's cells) and somatic variants (variants that arise during the lifetime of an individual). In one example, germline variants comprising SNPs and short indels are identified.

As illustrated in workflow 300, the filtered reference and patient sequencing files are input in the variant calling sub-process 325. Variant calling process 325 may use one or more different variant tools, or hybrid variant tools, to identify and process variants of interest in the sequencing files. Non-limiting examples of variant tools that may be used alone or in combination include: MuTect, Strelka, dysgu, JointSNVMix2, GATK, DRAGEN, or GATK-DRAGEN. In some instances, a hybrid variant tool integrating two or more variant tools together is used. In some instances, the hybrid variant tool integrates a variant caller into a genomic data analyzer. The variant caller portion of the hybrid variant tool may be a software-based tool (e.g., GATK) designed to analyze high-throughput sequencing data and be optimized in variant discovery. The variant caller may further process variant files in parallel by leveraging multi-threading and distributed computing. These processing styles allow tasks to be divided across multiple cores or nodes in a cluster to process large datasets faster than single-threaded tools. Moreover, the variant caller is designed to use streaming-based processing, which minimizes memory usage by processing the data (e.g., from BAM/CRAM files) in chunks instead of loading the entire dataset into the memory. This approach ensures that only the necessary subset of data is kept in memory at any given time.

The genomic data analyzer portion of the hybrid variant tool (e.g., DRAGEN) uses Field-Programmable Gate Array (FPGA) technology, which is specialized hardware designed to perform data processing tasks with extreme efficiency. Unlike traditional CPU- or GPU-based systems, FPGAs are highly parallelized and optimized for specific genomic algorithms, enabling faster data analysis with lower power consumption. This is in part due to FPGAs acting as hardware accelerators because they are configured to perform specific operations directly in hardware rather than relying on software execution. This eliminates the need for intermediate layers (e.g., operating systems or drivers) that can slow down processing. Additionally, because they perform the computations in hardware circuits rather than software, FPGAs have extremely low latency. FPGAs are semiconductor devices with three main components that allow them to process at ultra-fast speeds. The first component is configured logic blocks (CLBs) that perform logical and arithmetic operations (e.g., programmable logic gates, flip-flops, multiplexers, and lookup tables (LUTs)) that can be configured to execute specific functions. Importantly, each CLB can operate independently, meaning multiple tasks can be executed simultaneously. This parallelism is ideal for computationally intensive applications like genomic data analysis, where billions of data points must be processed concurrently. The second component is interconnects that link the configured logic blocks and other components together. This design allows data to flow efficiently between different blocks and be reprogrammed to suit the specific task at hand. The third major component of FPGAs is input/output blocks, which manage communication between the FPGA and external devices, such as storage systems, sensors, or other hardware.

In some instances, the variant tool (e.g., GATK) portion of the hybrid variant tool is used to aggregate STR data from the sequencing files (e.g., reference and patient sequencing files) into a table format that include information about the variant (e.g., locus information, sample information, quality metrics, etc.). The STR data table and the sequencing files are used as input for the genomic data analyzer portion of the hybrid variant tool (e.g., DRAGEN). The genomic data analyzer reconstructs the sequences in the STR table to determine the number of repeats for each allele at the STR locus and outputs a parameter file for each sample including information related to variant calling accuracy, error modeling, confidence scores, and the like. Variant calling process 325 can use these parameter files to guide other variant tools (e.g., variant calling tools) in handling regions of the genome that are complex in structure (i.e., STRs). Accordingly, variant calling process 325 may input the parameter files into a variant caller that identifies a subset of genetic variants. For example, the variant caller may specifically identify SNPs and indels. As such, genomic variant call files (e.g., GVCF) with SNP and indel calls for each reference sample and the patient sample are output from the variant calling process 325.

Individual reference variant files (e.g., GVCFs) output from the variant calling process 325 are input into a consolidation tool 330. The purpose of the consolidation tool 330 is to take the individual reference GVCF files and consolidate them by chromosome to produce a datastore formatted file for each chromosome that include all the SNPs and indels called for a particular chromosome from each reference sample. For example, consolidation tool 330 will output a file for each chromosome (e.g., 22 autosomal chromosomes) that contain the variant calls from all the reference samples. The consolidation tool 330 can use functions designed to organize GVCF data by genomic intervals (e.g., chromosomes, indexing) and output a datastore formatted file (e.g., a directory type file) containing the database files that can be queried by other tool functions.

Datastore formatted files are specialized data storage formats designed to improve the efficiency, scalability, and speed of data processing, particularly for large-scale datasets. This is achieved by leveraging partitioned storage, efficient indexing, columnar data organization, and distributed computing techniques. Data partitioning is a technique used to divide data into smaller, more manageable chunks based on genomic intervals (e.g., chromosome or genomic regions). Partitioning allows tools to process only the relevant portions of the dataset, reducing computational overhead and speeding up the workflow. The use of an efficient indexing mechanism enables rapid querying of specific genomic regions. Instead of scanning the entire dataset, tools can quickly locate and retrieve the data for targeted analysis. By storing the data in columns, instead of rows, processing tools can access specific attributes or fields (e.g., genotype likelihoods or annotations) faster. Moreover, only the relevant columns are loaded into memory during analysis, reducing memory usage and speeding up processing. Finally, the use of distributed computing frameworks allows the portioned data to be processed in parallel across multiple nodes, significantly reducing overall runtime for large datasets.

Following consolidation, the datastore formatted file, or individual GVCF files, may be processed by the joint variant calling tool 335 to generate a single variant file per chromosome with final variant calls for all the reference samples. To accomplish this, joint variant calling tool 335 combines the likelihoods and annotations for the samples at each genomic position in the reference sample datastore formatted file/GVCF files. For example, if multiple samples share the same variant at a given genomic location, the variant is recorded once in the joint variant file, with annotations for which samples carry the variant. In so doing, the most likely genotype for each sample at each site can be determined along with quality metric data (e.g., confidence scores, depth of coverage, allele frequency, etc.). all while reducing data storage requirements. Essentially, the joint calling tool 335 combines data from multiple samples to improve the accuracy of variant calls and consolidates repetitive calls into a single representative call.

The final variant files output from the joint variant calling tool 335 and the patient variant files output from variant calling 325 are input into a post-variant calling process 340 to assess genetic relatedness. Post-variant process 340 may perform some data pre-processing steps such as combining the chromosome variant files, sorting them, indexing them, file type conversions, etc. to organize the data in such a way that it can be analyzed by various software tools. Given the volume of data, the post-variant calling process 340 is designed to have high-performance computing power and uses efficient data compression and loading techniques, reducing memory usage for large datasets. In some instances, autosome and biallelic SNPs and indels may be extracted from the variant files. Post variant processing 340 outputs a reference variant file 345 comprising variants identified from the reference samples originally accessed from the data repositories 320. Also output from post-variant processing 340 is a patient variant file 355.

Patient sample variants 355 are merged with reference sample variants 345 to generate a merged variant file 360. During the merging process, some data cleaning can occur to resolve any merging errors, missing data (e.g., genotypes), variant filtering (based on minor allele frequency (MAF)), and the like to ensure the quality of the data. As a result, any detected pathogenic somatic mutations (which are only seen in the patient, resulting in a very low MAF), and problematic SNPs are removed before ancestry inference. Additionally, the variants in the merged variant filed 360 may be pruned for linkage disequilibrium. The merged variant file 360 is used as input into the genetically inferred ancestry calling pipeline 315 to determine genetic relatedness. As described herein, genetic relatedness quantifies the degree to which two individuals share genetic material inherited from a common ancestor. Methods for determining genetic relatedness include, but are not limited to, identical-by-descent (IBD), identical-by-state (IBS), kinship coefficient, relatedness coefficient (r), principal component analysis (PCA), co-ancestry coefficient (f), KING-robust coefficients, runs of homozygosity (ROH), polygenic scores for relatedness, haplotype sharing, or any combination.

The genetically inferred ancestry calling pipeline 315 implements three methods to derive GIA calls from the merged variant file 360. The methods include two different principal component-based classification methods (e.g., k-NN algorithm 365 and correlation-based algorithm 375) and admixture analysis 385.

### Principal Component Analysis

In some embodiments, a processing step comprises a principal component analysis (PCA). In some embodiments, sequence read counts (e.g., sequence read counts of a test sample) are adjusted according to a principal component analysis (PCA). In some embodiments a read density profile (e.g., a read density profile of a test sample) is adjusted according to a principal component analysis (PCA). A read density profile of one or more reference samples and/or a read density profile of a test subject can be adjusted according to a PCA. Removing bias from a read density profile by a PCA related process is sometimes referred to herein as adjusting a profile. A PCA can be performed by a suitable PCA method, or a variation thereof. Non-limiting examples of a PCA method include a canonical correlation analysis (CCA), a Karhunen-Loève transform (KLT), a Hotelling transform, a proper orthogonal decomposition (POD), a singular value decomposition (SVD) of X, an eigenvalue decomposition (EVD) of XTX, a factor analysis, an Eckart-Young theorem, a Schmidt-Mirsky theorem, empirical orthogonal functions (EOF), an empirical eigenfunction decomposition, an empirical component analysis, quasiharmonic modes, a spectral decomposition, an empirical modal analysis, the like, variations or combinations thereof. A PCA often identifies and/or adjusts for one or more biases in a read density profile. A bias identified and/or adjusted for by a PCA is sometimes referred to herein as a principal component. In some embodiments one or more biases can be removed by adjusting a read density profile according to one or more principal component using a suitable method. A read density profile can be adjusted by adding, subtracting, multiplying and/or dividing one or more principal components from a read density profile. In some embodiments, one or more biases can be removed from a read density profile by subtracting one or more principal components from a read density profile. Although bias in a read density profile is often identified and/or quantitated by a PCA of a profile, principal components are often subtracted from a profile at the level of read densities. A PCA often identifies one or more principal components. In some embodiments a PCA identifies a 1st, 2nd, 3rd, 4th, 5th, 6th, 7th, 8th, 9th, and a 10th or more principal components. In certain embodiments, 1, 2, 3, 4, 5, 6, 7, 8, 9, 10 or more principal components are used to adjust a profile. In certain embodiments, at least 20 principal components are used to adjust a profile. Often, principal components are used to adjust a profile in the order of appearance in a PCA. For example, where three principal components are subtracted from a read density profile, a 1st, 2nd and 3rd principal component are used. Sometimes a bias identified by a principal component comprises a feature of a profile that is not used to adjust a profile. For example, a PCA may identify a copy number alteration (e.g., an aneuploidy, microduplication, microdeletion, deletion, translocation, insertion) and/or a gender difference as a principal component. Thus, in some embodiments, one or more principal components are not used to adjust a profile. For example, sometimes a 1st, 2nd and 4th principal component are used to adjust a profile where a 3rd principal component is not used to adjust a profile.

A principal component can be obtained from a PCA using any suitable sample or reference. In some embodiments principal components are obtained from a test sample (e.g., a test subject). In some embodiments principal components are obtained from one or more references (e.g., reference samples, reference sequences, a reference set). In certain instances, a PCA is performed on a median read density profile obtained from a training set comprising multiple samples resulting in the identification of a 1st principal component and a 2nd principal component. In some embodiments, principal components are obtained from a set of subjects devoid of a copy number alteration in question. In some embodiments, principal components are obtained from a set of known euploids. Principal components are often identified according to a PCA performed using one or more read density profiles of a reference (e.g., a training set). One or more principal components obtained from a reference are often subtracted from a read density profile of a test subject thereby providing an adjusted profile.

k-NN

In some embodiments, a processing step comprises a k-nearest neighbors (k-NN) algorithm to classify a patient's genetic ancestry based on genetic relatedness to one or more ancestry populations. k-NN is a supervised machine learning model that works by finding the k closest data points (neighbors) in the training dataset (e.g., genetic PCs of reference samples) to a given query point (e.g., genetic PCs of the patient sample) and makes predictions based on their features and labels. In some embodiments, the k-NN algorithm 365 calculates the distance between the query point and every point in the training dataset using a distance metric (e.g., Euclidean distance, Manhattan distance, Minkowski distance, cosine similarity). Based on the calculated distances, the algorithm identifies the k closest data points (neighbors) to the query point. Then, the algorithm assigns the query point to the class that appears most frequently among the k nearest neighbors. For example, if k=8 (8 ancestry populations: AFR, AMR, CAS/SIB, EAS, EUR, MEA, OCN, an SAS), and the 8 nearest neighbors belong to classes (EUR, EUR, AMR, EUR, AMR, MEA, EUR, EUR) the prediction class by k-NN algorithm 365 would be EUR.

The principal component-based classification methods (e.g., k-NN algorithm 365 and correlation-based algorithm 375) may take as input the top 4, 5, 6, 7, 8, 9, 10, 15, 20 or more principal components to make a genetically inferred ancestry call. In certain embodiments, at least the top 20 principal components are used to make a genetically inferred ancestry call. The k-NN algorithm 365 may first be trained on the top genetic PCs (e.g., the top 20 PCs) of reference samples; then, the trained model is used to infer a first genetically inferred ancestry call 370. The correlation-based algorithm 375 calculates the Pearson correlations between a test sample (e.g., genetic PCs of the patient sample) and PCs of every reference sample. From these calculated correlations, the correlation-based algorithm 375 extracts a user defined top correlation percentage (e.g., top 1%) to predict a second genetically inferred ancestry call 380. The second genetically inferred ancestry call 380 is the reference population with the highest number of samples represented in the top correlations (see FIG. 7 for example visualizations of outputs). Additionally, the correlation-based algorithm can also produce a “Mixed Ancestry” call if two or more populations are included in equal numbers among the user defined top correlation percentage.

Admixture analysis method 385 is a model-based clustering algorithm, that estimates individual ancestries from genetic data (e.g., the merged variant file 360) using a maximum likelihood estimation. The admixture analysis method 385 can take as input a matrix, where the row represents a patient sample, the columns represent the genetic variants identified in the reference samples, and the cells specify the patient genotype at each variant. Using the input matrix, admixture analysis method 385 determines the frequencies of each allele at every marker in each ancestral population and the proportions of the patient's genome that originate from each ancestral population. From this analysis, a table is generated containing the percentage of the patient's genome originating from the populations that comprise the reference population dataset. The reference population reaching the largest admixture fraction output as the third GIA call 390.

Once the genetically inferred ancestry calling pipeline 315 generates the first GIA call 370, the second GIA call 380, and the third GAI call 390, a genetically inferred ancestry report 395 is generated based on the consensus of all three calls. In some instances, a consensus call is made when at least two out of the three calls are the same GIA for the patient sample. In some instances, if there is no alignment between the first GIA call 370 and the second GIA call 380 and all the third GIA calls 390 are <0.54, a GAI call of mixed ancestry is reported. In other instances, if there is no alignment between any of the methods and the third GIA call 390 is >0.54, the patient is given a consensus GIA of inconclusive.

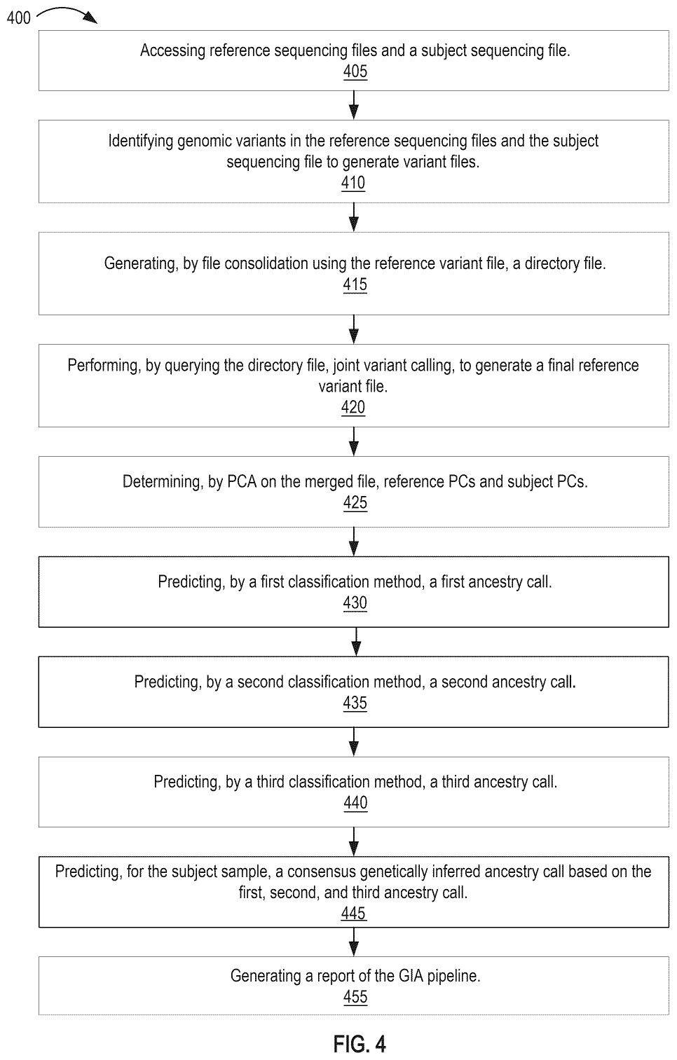

FIG. 4 shows a flowchart illustrating process 400 for predicting the genetically inferred ancestry of a patient in accordance with various embodiments. The processing depicted in FIG. 4 may be implemented in software (e.g., code, instructions, program) executed by one or more processing units (e.g., processors, cores) of the respective systems, hardware, or combinations thereof (e.g., the intelligent selection machine). The software may be stored on a non-transitory store medium (e.g., on a memory device). The method presented in FIG. 4 and described below is intended to be illustrative and non-limiting. Although FIG. 4 depicts the various processing steps occurring in a particular sequence or order, this is not intended to be limiting. In certain alternative embodiments, the steps may be performed in some different orders, or some steps may also be performed in parallel.

At box 405, reference sequencing files and a subject sequencing file are accessed. Both the reference sequencing files, and the subject sequencing file are generated as part of performing a NGS assay. The NGS assay can be whole-genome, exome, or targeted sequencing assays. In some instances, the reference sequencing files are generated from whole-genome or exome sequencing on a DNA sample isolated from a healthy tissue. In comparison, the subject sequencing file is generated by a targeted sequencing approach. More specifically, the subject sequencing file is generated from a comprehensive genome panel assay where the sequencing file comprises gene regions corresponding to the comprehensive gene panel used in the assay. In various instances, the comprehensive genome panel is a cancer genome panel, a non-invasive prenatal testing genome panel, or a transplant screening panel. The type of gene panel used is dependent on the type of testing the subject receives. For example, the subject may have cancer, in which case the comprehensive genome panel is a cancer genome panel (e.g., TruSight® Oncology 500). Alternatively, the subject may be a pregnant woman who is receiving noninvasive prenatal testing (NIPT), in which case the comprehensive genome panel may be Panorama®, Harmony®, or MaterniT21™. As another example, the subject may be in need of an organ transplant and is being assed for organ compatibility, in which case the comprehensive genome panel may be AlloSure® or Prospera™. In various embodiments, the subject sequencing file is generated from targeted (e.g., comprehensive genome panel) sequencing on a DNA sample isolated from a subject with cancer. More specifically, the DNA is tumor DNA. The sample taken from the subject may be acquired from a solid tumor biopsy or liquid biopsy (e.g., blood). The cancer may be Non-Small Cell Lung Cancer, Colorectal Cancer, Breast Cancer, Pancreatic Cancer, Head and Neck Cancer, Esophageal Cancer, Prostate Cancer, Neuroendocrine Tumors, Melanoma, Unknown Primary Cancer, Liver and Bile Duct Cancer, Stomach Cancer, Bladder Cancer, Kidney and Renal Pelvis Cancer, Sarcoma, Uterine Cancer, Ovarian Cancer, Small Intestine Cancer, or any other cancer known in the art.

The reference sequencing files, and subject sequencing file may be accessed from data repositories like data repositories 210 and 320 described with respect to FIG. 2 and FIG. 3 respectively. The reference sequencing files comprise individual sequencing files from different ancestral populations. The reference sequencing files comprise at least 1,000 individuals, at least 2,000 individuals, at least 3,000 individuals, or more from ancestral populations around the world. In various embodiments, at least 5, at least 6, at least 7, at least 8, or more ancestral populations are represented in the references sequencing files. In various embodiments at least 8 ancestral populations are represented in the reference sequencing files. The ancestral populations comprise African, European, Admixed American, East Asian, South Asian, Middle Eastern, Central Asian/Siberian, Oceania populations, or any combination thereof. In various embodiments, the ancestral populations consist of African, European, Admixed American, East Asian, South Asian, Middle Eastern, Central Asian/Siberian, and Oceania populations. Typically, five or fewer ancestral populations are used in conventional workflows often resulting in the misclassification of patient samples. For instance, individuals with Middle Eastern ancestry are frequently misclassified as European or South Asian in conventional workflows. By incorporating additional populations, the present workflow ensures more accurate and representative classifications for individuals across a wider range of ancestries without increasing computational time. Additionally, because the subject sequencing file comprises gene regions corresponding to a comprehensive genome panel, the reference sequencing files are filtered using the gene baits corresponding to the comprehensive gene panel used for the patient sample.

At box 410, genomic variants in the (filtered) reference sequencing files and the subject sequencing file are identified using a hybrid variant tool to generate reference variant files and a subject variant file. The genomic variants that can be identified in the filtered sequencing files include SNPs, indels, short tandem repeats (STRs), chromosomal rearrangements (e.g., translocations, inversions, and fusions), CNVs, and the like. Moreover, the genomic variants can comprise both germline variants (e.g., variants present in all the body's cells) and somatic variants (variants that arise during the lifetime of an individual). In one example, germline variants comprising SNPs and short indels are identified. In various instances, a hybrid variant tool integrating two or more variant tools together is used. In some instances, the hybrid variant tool integrates a variant caller into a genomic data analyzer. The variant caller portion of the hybrid variant tool may be a software-based tool (e.g., GATK) designed to analyze high-throughput sequencing by leveraging multi-threading and distributed computing techniques. Moreover, the variant caller is designed to minimize memory usage by processing data (e.g., from BAM/CRAM files) in smaller chunks instead of loading the entire dataset into the memory. The genomic data analyzer portion of the hybrid variant tool (e.g., DRAGEN) is a hardware-accelerated genomic analysis platform designed to deliver ultra-fast and accurate analysis of sequencing data. In various embodiments, the genomic data analyzer uses FPGA technology to perform specific operations (e.g., data table reconstruction) directly in hardware. Processing the (filtered) reference sequencing files and the subject sequencing file with the hybrid variant tool reduces the runtime of genomic analysis pipelines from hours to minutes, making it particularly valuable in clinical or large-scale research settings. In various embodiments, the variant caller portion of the hybrid variant tool performs variant calling using a likelihood-base comparison to determine the probability a variant exists in a given location. As such, genomic variant call files (e.g., GVCF) with SNP and indel calls for each reference sample and the patient sample are output.

At box 415, the reference variant files are consolidated and organized to generate a directory file. Consolidation happens on a per chromosome basis, to generate chromosome files comprising the genomic variants identified across the individual sample files that make up of reference samples. The chromosome files are organized and stored in a datastore formatted file (e.g., a directory type file) that can be queried by other tool functions. Various consolidation techniques may be used to generate the datastore formatted file such as, without limitation, partitioned storage, efficient indexing, columnar data organization, and distributed computing techniques.

At box 420, joint variant calling is performed by querying the datastore formatted file from box 415 to generate a final reference variant file. Instead of calling variants for each reference sample independently, joint variant calling aggregates genotype likelihoods and evidence (from the datastore formatted file) across the reference samples to make more accurate variant calls. Then, the final reference variant file is merged with the subject variant file, from box 410, to generate a merged variant file. The merged variant file may be filtered to ensure any detected pathogenic somatic mutations and problematic SNPs are removed prior to ancestry inference. Filtering can include removing variants with minor allele frequency (MAF)>0.1% and using the Hardy-Weinberg equilibrium exact test p-value>1E⁻⁶. Additionally, the merged variant file is pruned for linkage disequilibrium.

At box 425 principal component (PC) analysis is performed on the merged variant file to determine reference PCs and subject PCs, wherein the reference PCs and the subject PCs are comprised of a top set of PCs. Principal component analysis is a versatile technique for reducing dimensionality, identifying patterns, and simplifying complex datasets. By transforming data into a smaller set of principal components, PCA enables efficient analysis and visualization without compromising much of the original information. In various embodiments, at least 20 principal components are used in the top set of PCs.

At box 430, the reference PCs and the subject PCs are used in a first classification process to predict a first ancestry call based on the correlations found between the reference PCs and subject PCs. In various embodiments, the first classification method is a correlation-based algorithm that calculates the Pearson correlation between genetic PCs of the subject sample and PCs of every reference sample, extracts the top 1% of calculated correlations, and predicts the subject's ancestry population to be the reference population with the highest number of samples represented in the top correlations. In some embodiments, the correlation-based algorithm outputs more than one ancestry call if two or more populations are included in equal numbers among the top 1% of correlations.

At box 435, the reference PCs and the subject PCs are used in a second classification process to determine a second ancestry call based on a distance metric of the second classification process. In various embodiments, the second classification method is a k-nearest neighbor algorithm trained on the top set of PCs to predict the second ancestry call, and wherein the second ancestry call is the reference population that appears the most frequently in the k nearest neighbors. A k-nearest neighbors (KNN) algorithm is a non-parametric, supervised learning classifier, which uses proximity to make classifications or predictions about the grouping of an individual data point (e.g., the subject sample). In other words, a class label (ancestry call) is assigned to the individual data point on the basis of a majority vote (i.e. the label that is most frequently represented around the individual data point is used).

At box 440, the merged variant file is used in a third classification process to determine a third ancestry call based on a maximum likelihood estimation of the third classification process. In various embodiments, the third classification method is an admixture method, and the third ancestry call is the reference population with the highest ancestry fraction. Admixture analysis uses the merged variant file to determine the frequencies of each allele at every marker in each ancestral population and the proportions of the patient's genome that originate from each ancestral population. From this analysis, the percentage of the patient's genome originating from an ancestral population is learned. The population reaching an admixture fraction of greater than or equal to 0.54 (majority of ancestry plus an additional 0.04 to account for noise) is considered the third ancestry call.

At box 445, a consensus genetically inferred ancestry (GIA) call is predicted for the subject sample based on the first ancestry call, the second ancestry call, and the third ancestry call. The predicted consensus GIA call is reported as: (i) an ancestry type when at least two of either the first, the second, or the third ancestry calls are the same, (ii) mixed ancestry when all the maximum likelihood estimations from the third classification process are below a threshold, or (iii) inconclusive when no concordance across the first, second, and third ancestry calls and all the maximum likelihood estimations from the third classification process are below a threshold. In various embodiments, the threshold for the maximum likelihood estimations is less than 0.54.

Finally, at box 450, a report of the GIA pipeline is output. The report comprises the first ancestry call, the second ancestry call, the third ancestry call (e.g., ancestry fractions), and the consensus GIA call. Additional files that may be produced include a log file for the processes performed, eigenvectors and eigenvalues of patient and reference samples from PCA, training evaluations and performance of the k-NN model, plots of the top 20 principal components visualizing where the patient falls among reference samples and correlations of patient principal components with reference samples, and allele frequencies for each SNP in each reference population that was calculated by the admixture analysis. The report may be shared with the patient, a physician, a clinical researcher, or the like. In so doing, the report provides crucial data points to enable ancestry-aware biomarker research, which can help mitigate cancer outcome disparities, ensure inclusion of underrepresented groups in clinical research, and help findings be more representative of real-world patient populations who are eligible for targeted therapies or clinical trials.

### EXAMPLES

The following examples are offered by way of illustration, and not by way of limitation. The below examples are provided with respect to the sequencing results from the TruSight® Oncology 500 (TSO 500) CGP assay within TSO 500 probe regions. However, one of ordinary skill in the art can appreciate the ease to which the workflow may be extended to other CGP assays, allowing accurate GIA calls to be made across CGP assays.

Disparities in cancer diagnosis, treatment, and outcomes based on self-identified race and ethnicity (SIRE) are well documented, yet these variables have historically been excluded from clinical research. Without SIRE, genetic ancestry can be inferred using single-nucleotide polymorphisms (SNPs) detected from tumor DNA using comprehensive genomic profiling (CGP). However, factors inherent to CGP of tumor DNA increase the difficulty of identifying ancestry-informative SNPs, and current workflows for inferring genetic ancestry from CGP need improvements in key areas of the ancestry inference process. The workflow described herein used genomic data from 4,274 diverse reference subjects and CGP data from 491 patients with solid tumors and SIRE to obtain accurate genetically inferred ancestry (GIA) from CGP sequencing results. Consensus-based classification was used to derive confident ancestral inferences from an expanded reference data set covering eight world populations (African, Admixed American, Central Asian/Siberian, European, East Asian, Middle Eastern, Oceania, South Asian). The GIA calls were highly concordant with SIRE (95%) and aligned well with reference populations of inferred ancestries. Further, the workflow described herein expand on SIRE by (i) detecting the ancestry of patients that usually lack appropriate racial categories, (ii) determining what patients have mixed ancestry, and (iii) resolving ancestries of patients in heterogeneous racial categories and who had missing SIRE. Accurate GIA provides needed information to enable ancestry aware biomarker research, ensure the inclusion of underrepresented groups in clinical research, and increase the diverse representation of patient populations eligible for precision medicine therapies and trials.

### Example 1: Reference Sample Processing and Date Generation

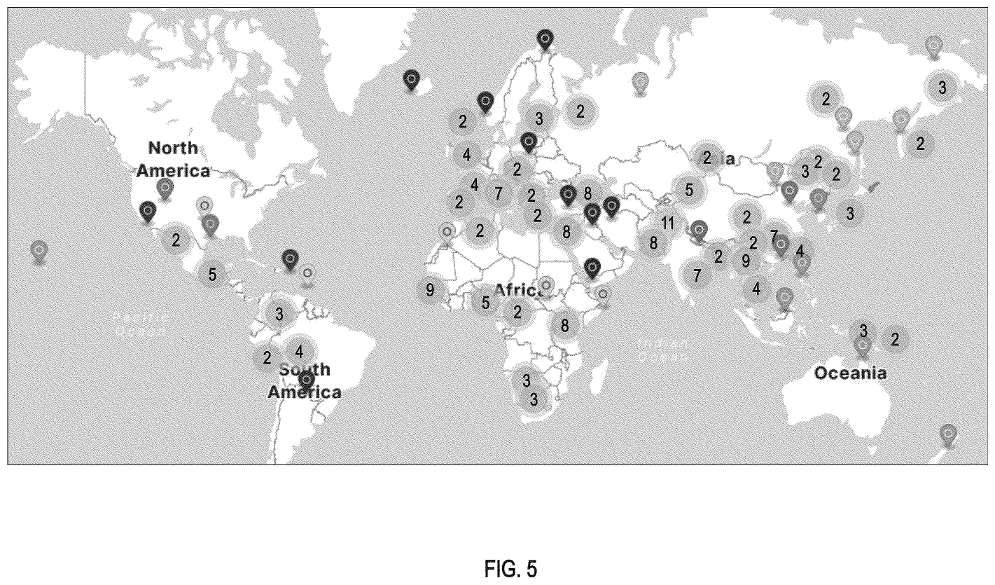

To perform genetic ancestry inference, SNP data derived from a large, diverse reference dataset with known ancestry is needed. The most used reference dataset for ancestry inference is the 1000G project data, which currently includes deep (30×) whole-genome sequencing of lymphoblastoid cell lines from 3,202 individuals covering five major geographical populations around the world and is now part of the International Genome Sample Resource. Populations covered by 1000G include African (AFR), Admixed Ameri-can (AMR; mainly Native Central and South American ancestry), East Asian (EAS), European (EUR), and South Asian (SAS) populations. Two additional diverse datasets included in the IGSR are the Human Genome Diversity Project (HGDP) and the Simons Genome Diversity Project (SGDP). The HGDP and SGDP datasets together includes 1,072 deeply sequenced individuals (35×-43× on average) from the same five geographical populations as 1000G and, additionally, Middle Eastern (MEA), Central Asian/Siberian (CAS/SIB), and Oceania (OCN) populations. Including these datasets added 811 additional reference samples to the pre-existing 1000G populations and uniquely enabled inference of MEA, CAS/SIB, and OCN ancestry, which have yet to be included in previous workflows. A summary of the reference datasets and included populations used in the workflow described herein can be found in Table 1 along with total reference sample numbers pre- and postprocessing. Additionally, FIG. 5 shows a map of geographical populations included as reference samples in the GIA workflow.

TABLE 1
Whole-genome sequenced reference samples used in the GIA workflow.

| | Datasets | | | Total pre- | Total post- |
|---|---|---|---|---|---|
| Populations | 1000G | HGDP | SGDP | processing | processing |
| African | 893 | 88 | 35 | 1016 | 751 |
| Admixed American | 490 | 51 | 22 | 563 | 423 |
| East Asian | 585 | 170 | 51 | 806 | 728 |
| European | 633 | 137 | 43 | 813 | 705 |
| South Asian | 601 | 181 | 35 | 817 | 728 |
| Middle Eastern | 0 | 153 | 23 | 176 | 175 |
| Central Asian/Siberian | 0 | 23 | 27 | 50 | 50 |
| Oceania | 0 | 25 | 8 | 33 | 32 |
| All populations | 3202 | 828 | 244 | 4274 | 3592 |

1000G, 1000 Genomes; HGDP, Human Genome Diversity Project; SGDP, Simons Genome Diversity Project. The reduction of reference sample size from pre- to postprocessing was due to the exclusion of samples from related individuals.

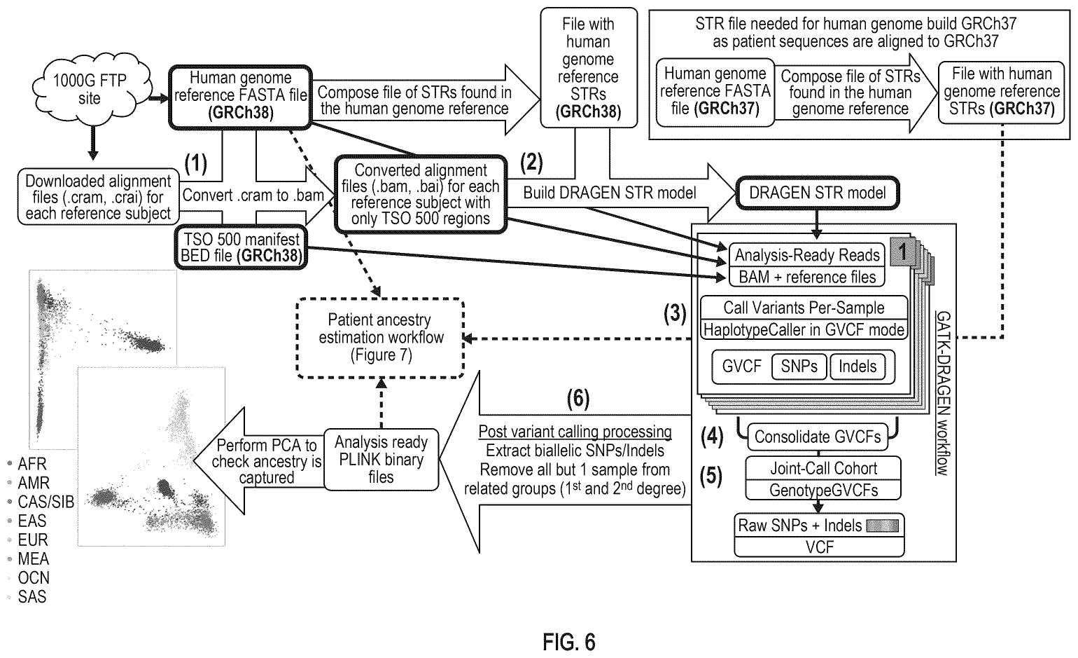

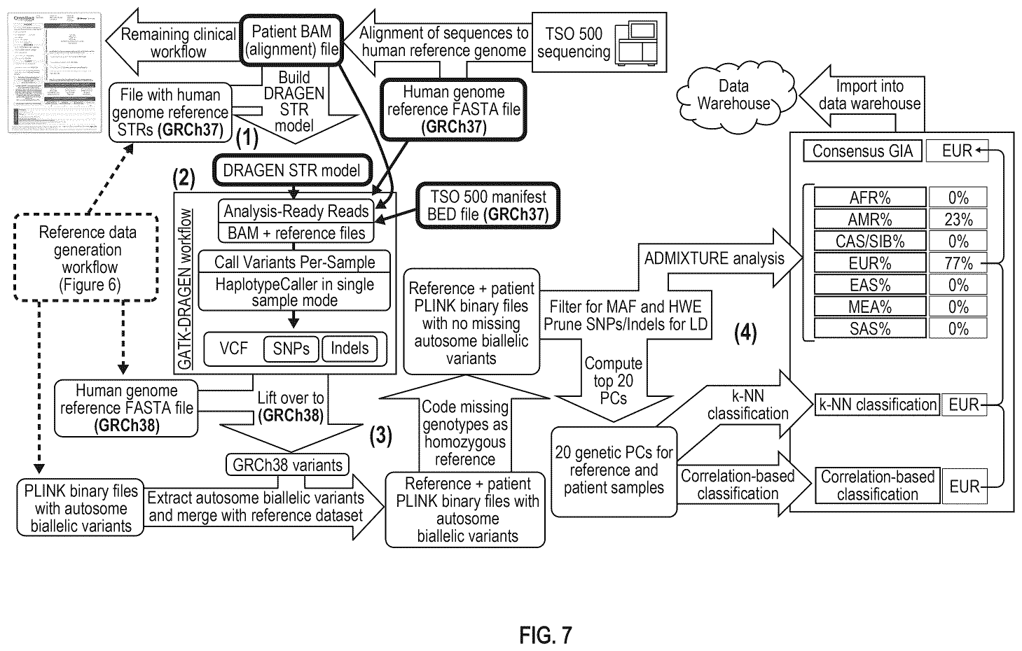

A flowchart showing the general steps taken for reference sample processing and data generation is provided in FIG. 6. Compressed Reference-oriented Alignment Map (CRAM) files for the 1000G dataset and the HGDP and SGDP datasets were obtained. Germline variant calling of SNPs and short insertion/deletions (Indels) utilizing GATK-DRAGEN was performed for reference samples using samtools and GATK, targeting regions covered by the TSO 500 bait set. Germline variant calling involved the following steps: (i) CRAM to Binary Alignment Map (BAM) file conversion, (ii) DRAGEN short tandem repeat (STR) model construction (required when running GATK-DRAGEN), (iii) sample-level variant calling with GATK-DRAGEN, (iv) consolidation of single-sample variant calls per chromosome, (v) joint variant calling, and (vi) post variant call processing. Details on how each step was performed are given below.

(1) CRAM to BAM file conversion. To convert CRAM files to BAM files for all datasets targeting only TSO 500 regions, the command ‘samtools view -b -T $GRCh38.fa -M -L$regions -write-index -o $file name.bam $file name.cram’ was used where $file name.cram was the input CRAM file of a reference sample, $regions was the TSO 500 manifest obtained directly from Illumina (San Diego, CA) that lists bait set regions in BED format, and $GRCh38.fa was the GRCh38 human genome reference used to create the CRAM files.

(2) DRAGEN STR model construction. The STR model, required to run GATK-DRAGEN, was created using the command ‘gatk CalibrateDragstrModel -reference $GRCh38.fa -input$file name.bam -intervals $regions -interval-padding 200 -str-table-path $GRCh38.STR.zip -output DRAGEN STR model.txt’ where $GRCh38.STR.zip was a STR table file created with the GATK ComposeSTRTableFile function using the GRCh38 reference file as input.

(3) Sample-level variant calling with GATK-DRAGEN. SNP and indel calling was performed using GATK's HaplotypeCaller in DRAGEN mode by using the command ‘gatk HaplotypeCaller -reference $GRCh38.fa -input $file name.bam -intervals$regions -interval-padding 200 -output $file name.g.vcf.gz-emit-ref-confidence GVCF -dragen-mode true -dragstr-params-path DRAGEN STR model.txt’, producing a genomic variant call format (GVCF) file with SNP and indel calls for each sample.

(4) Sample-level variant call consolidation. Individual reference sample GVCFs were consolidated by chromosome with the command ‘gatk GenomicsDBImport -batch-size 50-bypass-feature-reader true -consolidate true -intervals$chr -genomicsdb-workspace-path $chr.db -reference$GRCh38.fa-sample-name-map gvcf.sample map’, producing a GenomicsDB datastore formatted file for each chromosome ($chr.db) that contains all SNPs and indels called for that chromosome from each reference sample.

(5) Joint variant calling of reference samples was performed for each chromosome using the command ‘gatk Genotype-GVCFs-reference $GRCh38.fa -variant gendb://$chr.db -output $chr.vcf.gz’, producing one VCF per chromosome with final variant calls for all reference samples.

(6) Post variant call processing. Per-chromosome VCFs were combined, sorted, and indexed using the ‘concat’, ‘sort’, and ‘index’ functions from bcftools. Using PLINK2, VCFs were converted to PLINK binary file formats keeping only autosome and biallelic variants. Genetic relatedness (measured via KING-robust coefficients) was calculated using PLINK2, with one reference sample from related groups retained (KING-robust coefficient>0.09375, i.e. first- and second-degree relations). African populations with observed high within-population genetic similarity (Mbuti, Biaka, Jul'hoan/San, and Bantu in South Africa and Tswana; N=52) were also removed before patient ancestry inference, as the inclusion of these populations skewed PCA results.

After variant calling and processing, SNP data for 3592 (84%) reference samples were available and incorporated into the GIA workflow.

### Example 2: Genetically Inferred Ancestry Workflow for Inferring Patient Ancestry from Comprehensive Genomic Profiling Sequencing Results

The GIA workflow described here (FIG. 7) was designed to take TSO 500 sequencing results within the validated OmniSeq® INSIGHT laboratory-developed test as input, beginning with a sample level DNA-sequence alignment file (GRCh37 build). From there, the GIA workflow includes four main steps: (i) DRAGEN STR model construction, (ii) sample-level variant calling with GATKDRAGEN, (iii) post-variant call processing and merging with reference dataset variants, and (iv) performing ancestry inference from patient-reference merged data. Details on GIA workflow steps are provided below.

(1) DRAGEN STR model construction. The STR model is created using the command ‘gatk CalibrateDragstrModel -reference $GRCh37.fa -input $file name.bam -intervals $regions -interval-padding 200 -str-table-path $GRCh37.STR.zip -output DRAGEN STR model.txt’ where $file name.bam is the BAM file with aligned tumor sequences from sequencing with TSO 500, $GRCh37.fa is the human reference genome FASTA file (as TSO 500 sequences are aligned with this genome build by default), $GRCh37.STR.zip is a STR table file created with the GATK ComposeSTRTableFile function using the GRCh37 reference file as input, and $regions are the TSO 500 bait regions in GRCh37 coordinates.

(2) Sample-level variant calling with GATK-DRAGEN. Commands for sample-level variant calling are the same as those used for calling variants of reference samples with the following modifications: GRCh37 reference genome and STR table files are used in place of GRCh38 files, patient sample level variant calling is performed per chromosome to speed up processing, and no joint variant calling is performed as the workflow is performed for one patient sample at a time.

(3) Post variant call processing and merging with reference dataset variants. Like processing of reference samples, per chromosome VCFs are combined, sorted, and indexed using the ‘concat’, ‘sort’, and ‘index’ functions from bcftools and then converted to PLINK binary file formats keeping only autosome and biallelic variants. However, before converting to PLINK binary files, to harmonize variant positions between patient variants (GRCh37 build) and reference variants (GRCh38 build), positions are updated to GRCh38 using the ‘Liftover Vcf’ function from the Picard suite of tools. Patient sample variants were merged with reference sample variants using the ‘bmerge’ function in PLINK (function not fully implemented in PLINK2 at the time of writing), automatically resolving any merging errors; then, missing genotypes are given the genotype of homozygous reference allele to avoid batch effects due to missingness. Lastly, using PLINK2, merged patient-reference variant data are filtered for variants with minor allele frequency (MAF)>0.1% and Hardy-Weinberg equilibrium exact test P-value>1E⁻⁶ using the “midp” modifier to apply a mid-p adjustment to the exact test P-values. This filtering step helps to ensure any detected pathogenic somatic mutations (which will only be seen in the patient, resulting in a very low MAF), and problematic SNPs are removed before ancestry inference.

(4) GIA calling and consensus determination. Three methods are used to derive GIA calls from the merged patient-reference variant data: two different principal component (PC)-based classification methods and admixture analysis. Then, a consensus GIA is derived from the outputs of all three methods.

For all three methods, variants are first pruned for linkage disequilibrium (LD) using PLINK2's ‘indeppairwise’ function with a 100 variant sliding window and R² threshold of 0.2.

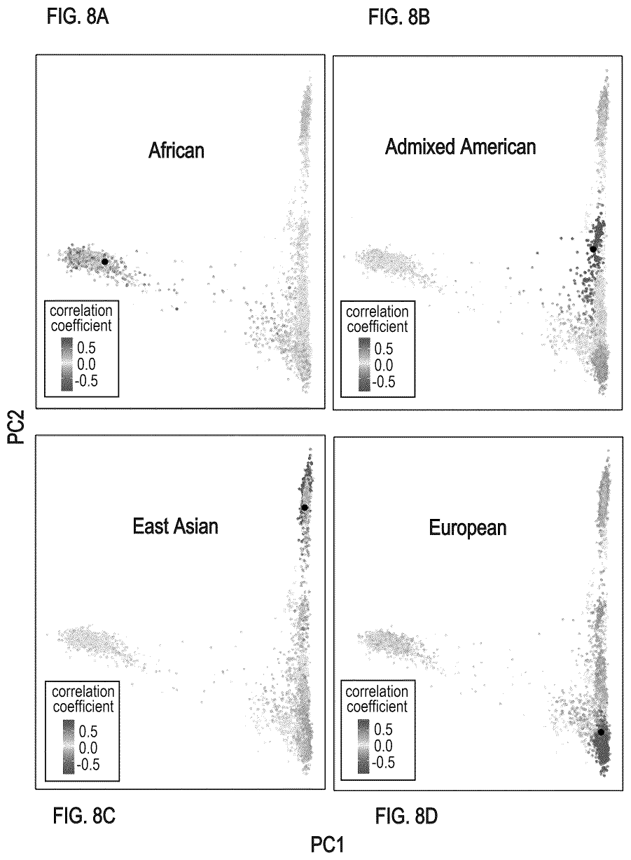

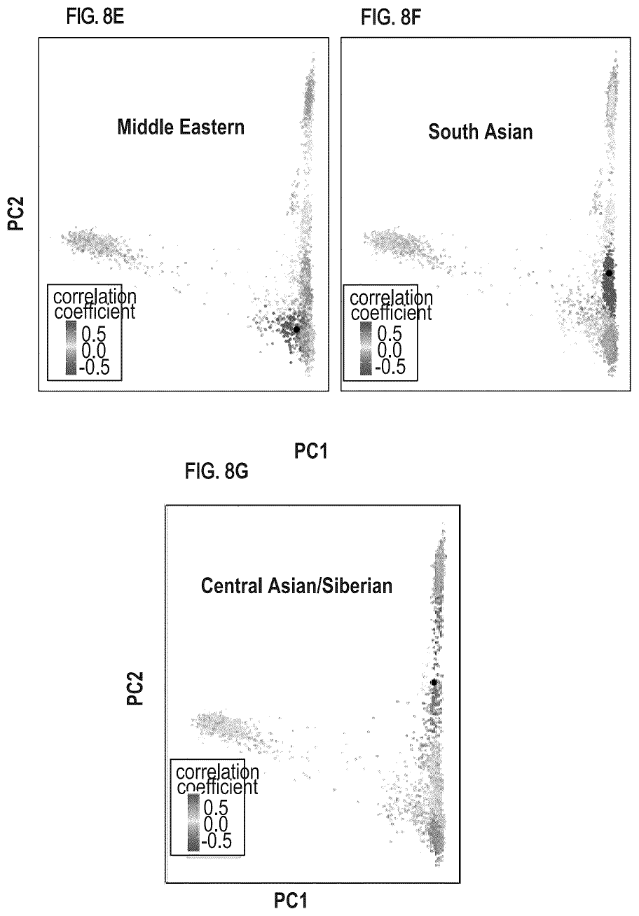

For PC-based methods, LD-pruned variants are used to calculate the first 20 genetic PCs (via PLINK2) for patient and reference samples. The top 20 genetic PCs are used as input to two classification methods implemented in R and a custom correlation-based algorithm. The k-NN algorithm is first trained on genetic PCs of reference samples; then, the trained model is used to infer an ancestry population for the patient. The correlation-based algorithm calculates the Pearson correlation between genetic PCs of the patient sample and PCs of every reference sample (3592 correlations), extracts the top 1% of calculated correlations, and predicts the patient's ancestry population to be the reference population with the highest number of samples represented in the top correlations (FIGS. 8A-8G). The outputs from PC-based classifications are two discrete GIA calls (one of AFR, AMR, CAS/SIB, EAS, EUR, MEA, OCN, or SAS from k-NN and correlation-based classification). The correlation-based algorithm can also produce a “Mixed Ancestry” call if two or more populations are included in equal numbers among the top 1% of correlations.

For admixture analysis, LD-pruned variants are provided directly to the program ADMIXTURE along with a population file listing the reference populations and a hyphen for the patient (symbolizing the ancestry is unknown). Admixture analysis is performed using ADMIXTURE's ‘--supervised’ flag and setting k to equal the number of reference populations. The output from ADMIXTURE includes predicted ancestry fractions of a patient being contributed to by each reference population. The population reaching an admixture fraction of >0.54 (majority of ancestry plus an additional 0.04 to account for noise) is considered the discrete GIA call when determining a consensus GIA call. Of note, data for Oceania reference samples (N=32) are removed before running ADMIXTURE (hence k=7) due to a bias noted during testing where ADMIXTURE would estimate every patient to have a fraction of Oceania ancestry, masking ancestry fractions from other populations. This bias might result from lower heterozygosity and higher LD previously shown in New Guinea-based Oceania populations that make up much of the Oceania reference population used here. Oceania ancestry estimation is still made possible, however, through PC-based classification.

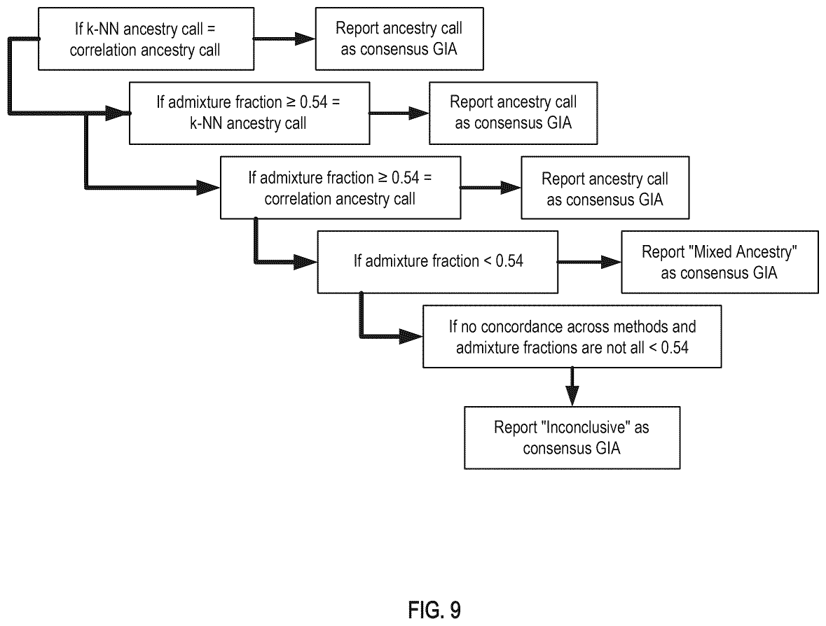

To derive a consensus call, two out of three methods are required to call the same GIA for a patient sample (FIG. 9). If there is no alignment between the two PC-based classifications and all ancestry fractions are <0.54, the patient is given a consensus GIA call of “Mixed Ancestry.” If there is no alignment between any of the methods when a majority ancestry fraction is present for ADMIXTURE results, the patient is given a consensus GIA of “Inconclusive”; however, the individual method results are still included in the final output.

PC-based GIA calls, admixture fractions, and the consensus GIA call for a patient are the final outputs of the workflow.

### Example 3: TruSight R Oncology Sequencing of the Technical Validation Cohort

DNA and RNA were co-extracted from formalin-fixed paraffin embedded (FFPE) tissue specimens and submitted for library preparation and sequencing using the hybrid-capture-based TSO 500 assay (Illumina, San Diego, CA) as part of OmniSeq® INSIGHT (OmniSeq, Buffalo, NY). Within the TSO 500 assay, DNA sequencing with hybrid capture was used to detect small nucleotide variants in exonic regions of 523 genes (single and multi-nucleotide substitutions, insertions, and deletions) and copy number variants in 59 genes (gains and losses), as well as analysis of microsatellite instability (MSI) and tumor mutational burden (TMB) genomic signatures. RNA sequencing with hybrid capture detects fusions and splice variants in 55 genes. Only DNA sequencing results were used as input to the GIA workflow for deriving GIA calls of the validation cohort.

### Example 4: Technical Validation of GIA Workflow

GIA was determined for a validation cohort of 504 patients who underwent CGP testing via TSO 500 at a reference laboratory (OmniSeq/Labcorp, Buffalo, NY, USA) during standard care (Table 2).

TABLE 2
Validation cohort characteristics

| Variable | N | Summary stats |
|---|---|---|
| Total number of patients | 504 | — |
| Self-reported race and ethnicity (N, %) | 491 | |
| [Given 1000 Genomes population] | | |
| White [EUR] | | 367 (74.7%) |
| Black or African American [AFR] | | 82 (16.7%) |
| Hispanic or Latino [AMR] | | 19 (3.9%) |
| American Indian or Alaska | | 20 (4.1%) |
| Native [AMR] | | |
| Asian-Indian [SAS] | | 2 (0.4%) |
| Asian-Vietnamese [EAS] | | 1 (0.2%) |
| Sex (N, %) | 504 | |
| Female | | 233 (46.2%) |
| Male | | 271 (53.8%) |
| Age, years (Mean ± SD) | 504 | 68.5 ± 12 |
| Cancer type (N, %) | 504 | |
| Non-Small Cell Lung Cancer | | 138 (27.4%) |
| Colorectal Cancer | | 79 (15.7%) |
| Breast Cancer | | 57 (11.3%) |
| Pancreatic Cancer | | 33 (6.5%) |
| Head and Neck Cancer | | 24 (4.8%) |
| Esophageal Cancer | | 22 (4.4%) |
| Prostate Cancer | | 22 (4.4%) |
| Neuroendocrine Tumors | | 19 (3.8%) |
| Melanoma | | 16 (3.2%) |
| Stomach Cancer | | 16 (3.2%) |
| Unknown Primary Cancer | | 15 (3%) |
| Liver and Bile Duct Cancer | | 11 (2.2%) |
| Bladder Cancer | | 8 (1.6%) |
| Kidney and Renal Pelvis Cancer | | 7 (1.4%) |
| Sarcoma | | 7 (1.4%) |
| Uterine Cancer | | 6 (1.2%) |
| Cervical | | 5 (1%) |
| Ovarian Cancer | | 5 (1%) |
| Small Intestine Cancer | | 5 (1%) |
| Other Cancer | | 9 (1.8%) |
| Known clinical stage (N, %) | 385 | |
| Stage II | | 3 (0.8%) |
| Stage III | | 109 (28.3%) |
| Stage IV | | 273 (70.9%) |
| Tumor specimen location (N, %) | 490 | |
| Metastatic | | 164 (33.5%) |
| Primary | | 326 (66.5%) |
| TMB (mutations/Mb) (Mean+SD) | 484 | 13.5 ± 28.4 |
| TMB level (N, %) | 484 | |
| High (≥10) | | 138 (28.5%) |
| Not high (<10) | | 346 (71.5%) |
| MSI level (N, %) | 489 | |
| MSI High | | 13 (2.7%) |
| Stable | | 456 (97.3%) |
| Number of neoplastic cells | 504 | |
| per slide (N, %) | | |
| <1000 | | 120 (23.8%) |
| ≥1000 | | 112 (22.2%) |
| ≥2000 | | 272 (54%) |
| Tumor specimen cellularity (N, %) | 504 | |
| ≤2 | | 395 (78.4%) |
| >2 | | 109 (21.6%) |

N, number of patients with data;
SD, standard deviation;
AFR, African;
AMR, Admixed American;
EAS, East Asian;
EUR, European;
SAS, South Asian;
TMB, tumor mutational burden;
MSI, microsatellite instability.

Data for 484 patients (96%) were collected as part of the PREFER (PRospective registry oF advanced stage cancER) clinico-genomic patient registry. Patients with advanced-stage solid cancers consented to participate in PREFER from multiple oncology practices focused on serving underrepresented populations. The remaining 20 patients underwent CGP testing during standard care at an Alaskan-based facility that serves native Alaskan populations and were included to provide cases with American Indian or Alaska Native race, which were lacking in the PREFER registry. Of the 504 patients, 491 (97.4%) had available SIRE data to compare with GIA calls. These patients were assigned a reference population (one of AFR, AMR, CAS/SIB, EAS, EUR, and SAS) based on their SIRE data to provide a direct comparison to GIA calls (Table 2). Middle Eastern and Oceania populations were not assigned to patients as there were no appropriate racial or ethnic categories reported for these populations. The remaining 13 patients (2.6%) had unknown or missing SIRE data but were still included in ancestry inference as an example of how GIA can resolve missing SIRE data. Additionally, three patients had sequencing performed twice on the same tumor specimen (technical replicates), and six patients had sequencing performed on two different tumor specimens from two different tissues (biological replicates), which allowed us to assess the stability of GIA calls across sequencing runs on the same or different tumor specimens taken from different tissue locations. All calculations, analyses, and plotting for the technical validation of the GIA workflow were performed in R.

Several checks were performed independently of patients' SIRE to ensure the robustness of GIA calls. Differences in the number of aligned reads between called GIA groups were assessed using the Wilcoxon rank-sum test on log-transformed sequence read counts to ensure that GIA calls were not biased by the total number of sequence reads of a sample. The top two genetic PCs of patients were projected onto reference sample PCs to ensure that GIA calls of patients aligned with the reference group to which they were most genetically akin. To have one data point per reference sample for PC 1 and 2, the median was used as PCA was performed independently for each patient, resulting in many measures of PC 1 and 2 for each reference sample. Lastly, admixture fractions and their distributions within each GIA group were plotted to determine if called GIA groups had higher fractions of the appropriate populations.

GIA calls were compared to the SIRE of each patient to assess overall concordance (proportion of patients with matching GIA and SIRE) and classification performance metrics (sensitivity/re-call, specificity, balanced accuracy, precision, F1-score). Patients with inferred ancestries without SIRE information were counted in the calculations as “misclassified” to assess the extent to which GIA deviates from SIRE due to the added resolution of appropriate ancestral populations that do not have a matching racial or ethnic category reported. Concordances between GIA calls and SIRE were also assessed for significant differences across patient tumor types, tissue characteristics (primary versus metastatic sites, number of neoplastic cells per specimen slide, tumor specimen cellularity), and genomic characteristics (TMB low/high, MSI status, presence or absence of copy number alterations or gene fusions/rearrangements) using Fisher's exact test to ensure that these factors do not introduce any biases.

Classification performance metrics were calculated via the ‘confusionMatrix’ function in the caret R package, specifying the mode to be “prec recall.” Plotting of technical validation results was performed using ggplot2 and various packages to extend ggplot2 functionality. For any statistical analyses, uncorrected P-values<0.05 were considered statistically significant. All reported P-values were two-sided.

### Example 5: Technical Validation Results of the GIA Workflow: Validation Cohort Characteristics

The validation cohort used for assessing the performance of the GIA workflow included patient tumor samples from a spectrum of races and ethnicities, ages, cancer types, and genomic biomarker characteristics (Table 2). Most patients self-identified as White (74.7%) followed by Black or African American (16.7%), American Indian or Alaska Native (4.1%), Hispanic or Latino (3.9%), and Asian (0.6%). While “Asian” does not typically distinguish between East and South Asian individuals, patients who self-reported Asian race also included their ethnicities, so these patients were subclassify as Asian Indian (0.4%) or Asian Vietnamese (0.2%). Of note, while there were 20 patients with American Indian or Alaska Native for their race, 52 patients self-identifying as White (14.1% of White patients) reported their ethnicity as “Native American”. White (without Hispanic or Latino ethnicity), Black or African American, and American Indian or Alaska Native patients were given a reference population label of EUR, AFR, and CAS/SIB, respectively. Hispanic or Latino patients were given a reference population label of AMR. Typically, American Indian or Alaska Native-identifying individuals would be labeled as AMR along with Hispanic or Latino; however, the addition of the CAS/SIB reference group in the workflow allows us to distinguish between North and Central/South American-based Native American ancestry. Asian patients reporting an ethnicity of Indian or Vietnamese were given a reference population label of SAS or EAS, respectively. The validation patient cohort was relatively balanced for males and females. Most patients were older than 60 years with a mean age of 68.5±12. Patients with non-small lung cancer accounted for almost one-third of the dataset (27.4%), followed by colorectal (15.7%), breast (11.3%), pancreatic (6.5%), head and neck (4.8%), and 15 other cancer types comprising <5% of the cohort each. Most patient tumor tissue samples were collected from primary sites (66.5%), had low TMB (<10 mutations/Mb, 71.5%), and were microsatellite stable (97.3%).

### Example 6: Technical Validation Results of the GIA Workflow: GIA Workflow Results for the Validation Cohort

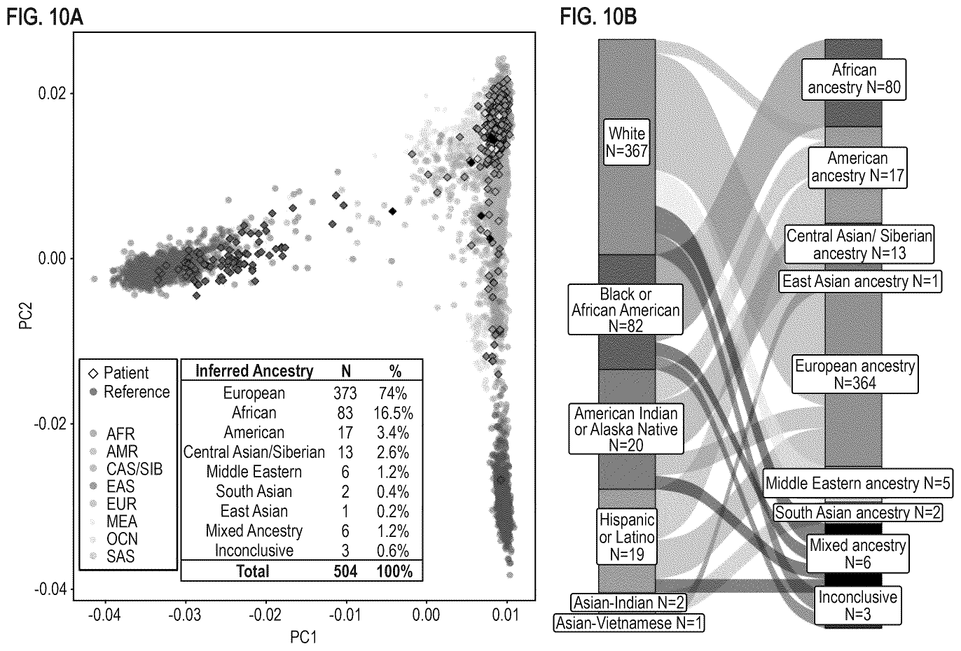

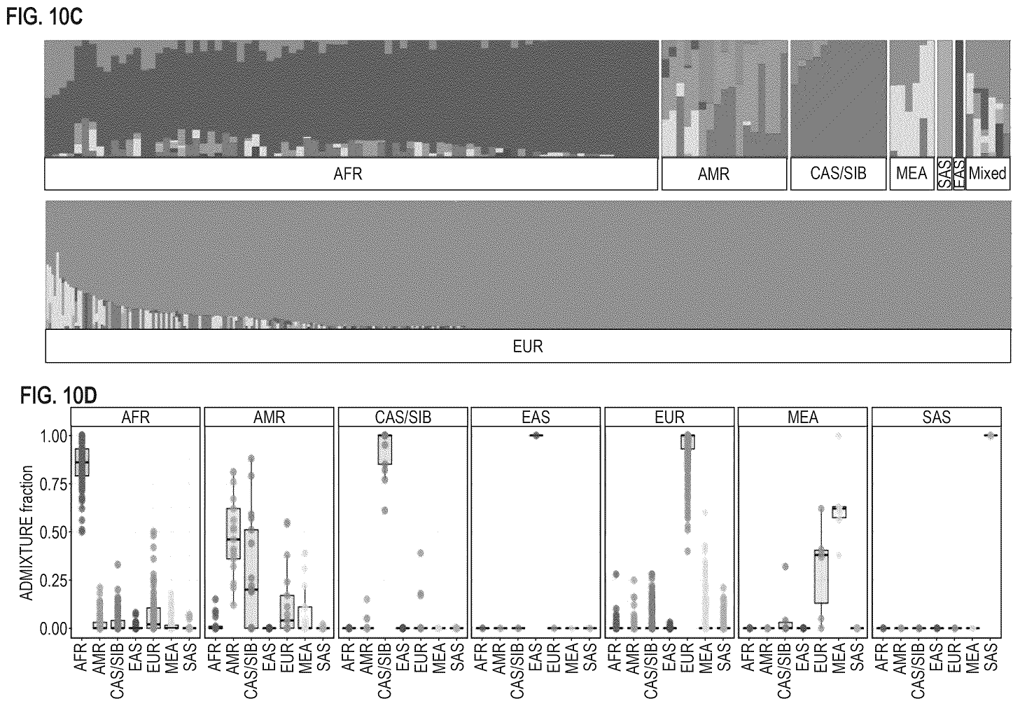

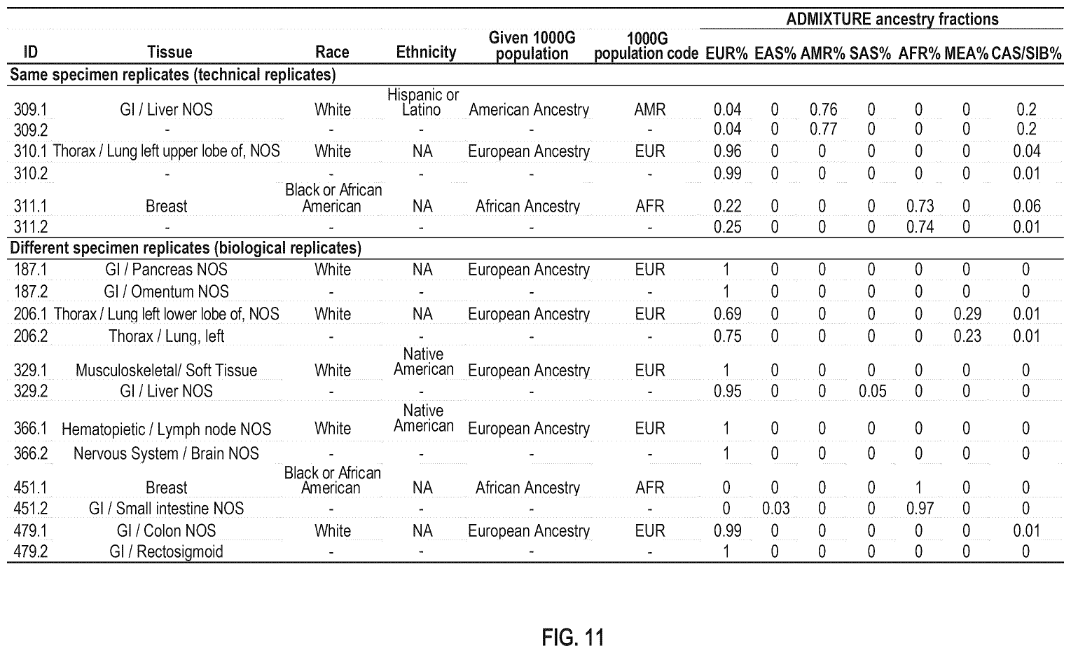

The GIA workflow was used to successfully obtain a consensus GIA for 501 patient samples (99.4%) from the validation cohort, while three patient samples were inconclusive for their consensus GIA (FIG. 10A). All three technical replicates and six biological replicates had 100% concordance in GIA calls with only slight fluctuations in their ancestral fractions estimated via ADMIXTURE. Tissue locations from which biological replicates were taken ranged from different areas of the same organ site (e.g. varying regions of the lung) to separate areas of the body (e.g. breast and small intestine) (FIG. 11), suggesting that reproducibility of GIA calls was not influenced by differences in tumor specimen tissue location.

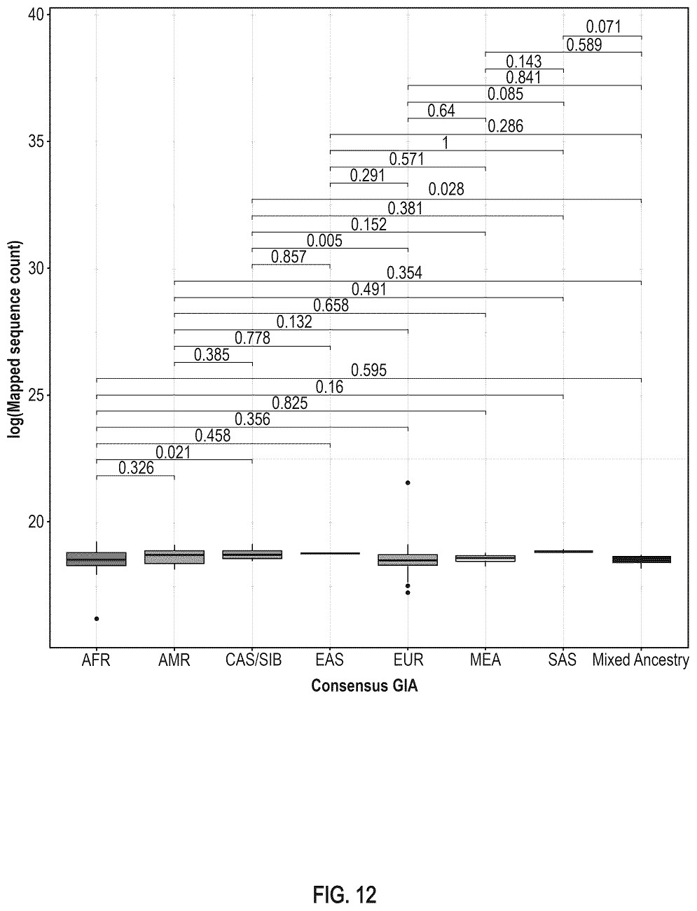

Overall, the average number of aligned sequence reads per patient BAM file was 117 million, and no outlying GIA groups were observed in the number of aligned reads (FIG. 12) showing that GIA calls overall were not biased by the total number of sequence reads of a sample. Failure to call a consensus GIA for three patient samples resulted from isolated instances of poor sequencing output, and therefore poor SNP calling, as sequence alignment files for patients with inconclusive GIA calls contained <12 000 sequences (8578 to 11 779 sequences) while the next-highest number of sequences was 11 million. Interestingly, all patient samples that resulted in an inconclusive consensus GIA had the same profiles among the three classification methods regardless of their SIRE: AMR for correlation-based classification, SAS for k-NN classification, and 100% CAS/SIB for admixture analysis, which suggests that inconclusive results, at least those derived from low sequencing and SNP output, can be consistently caught.

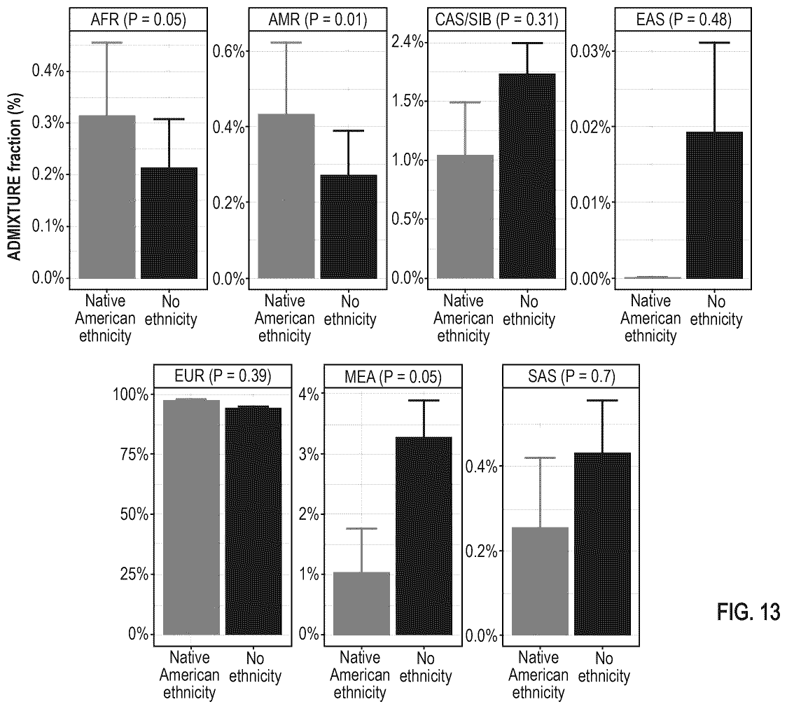

When projected onto reference sample genetic PCs (FIG. 10A), patient data points overlapped with reference samples from their inferred ancestral population, which suggests that GIA calls overall were made successfully with no “off-target” inferences. Patients' consensus GIA calls aligned with their SIRE and expanded upon SIRE by (i) differentiating between those of European and Middle Eastern ancestry who reported as “White” and (ii) detecting the presence of mixed ancestry (FIG. 10B). GIA calls were also able to resolve heterogeneous racial categories with high ancestral admixture (e.g. American Indian or Alaska Native and Hispanic or Latino categories) (FIG. 10B). Ancestral fractions calculated via ADMIXTURE mirrored the consensus GIA calls (FIG. 10C) with the highest ancestral fractions within each consensus GIA group corresponding to the inferred ancestral population (FIG. 10D). It was interesting to note that while the highest median ancestral fraction of the AMR GIA group was AMR ancestry, this group also had high fractions of CAS/SIB ancestry (FIG. 10D), which may reflect Central Asian/Siberian ancestry in Central/South American-based Native American populations. All patients who self-identified as White and reported Native American as their ethnicity resulted in consensus GIA calls of EUR and had overall lower ancestry fractions from the AMR (0.4%±1.3%) and CAS/SIB (2.9%±14.1%) populations compared to patients identifying as American Indian or Alaska Native (AMR=9.1%±15.8%, CAS/SIB=75.9%±30.9%) or His-panic or Latino (AMR=37.9%±27.6%, CAS/SIB=10.2%±17.8%). However, compared to White patients with no reported ethnicity, these patients had slightly, but significantly, higher AMR ancestry fractions (0.4% versus 0.3%, P=0.01; FIG. 13), suggesting that these patients potentially have Native American ancestry with higher proportions of European admixture.

### Example 7: GIA Call Classification Performance

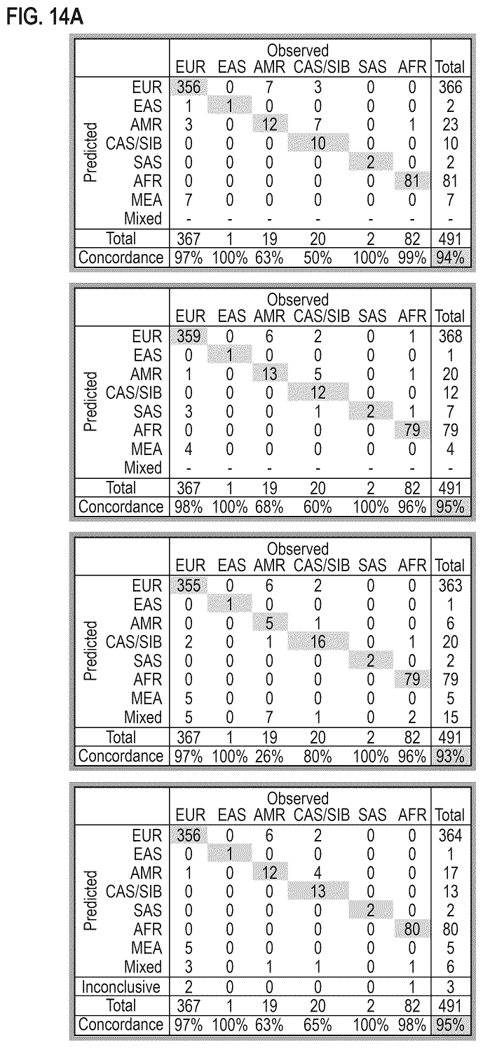

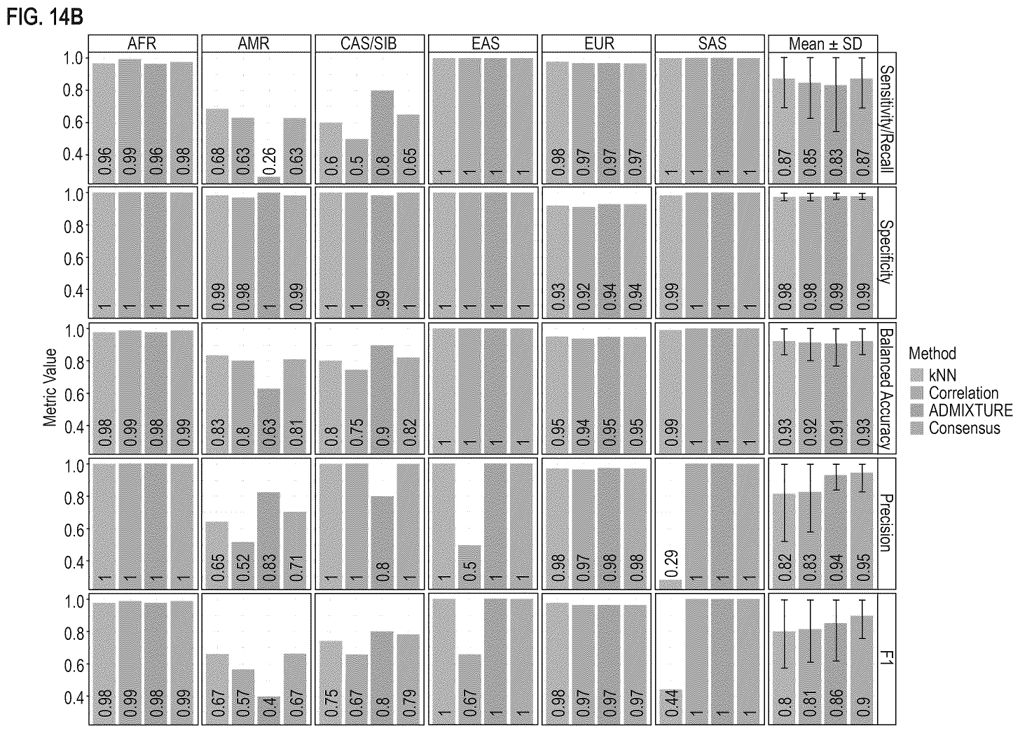

GIA calls showed high concordance with SIRE (93%-95% for individual methods and 95% for consensus GIA calls; FIG. 14A) and resulted in a high classification performance when using SIRE as the ground truth (FIG. 14B). Classification metrics across all reference populations ranged from 0.8 to 1 depending on the classification method, with consensus GIA calls ranging from 0.87 (sensitivity/recall) to 0.99 (specificity). The classification performance of consensus GIA was equivalent to, or better than, the independent methods in the study, with the largest improvement seen for the F1 score (≤0.86 for independent methods versus 0.9 for consensus). The lowest concordance between consensus GIA calls and SIRE was seen for patients reporting as Hispanic or Latino (63%) and American Indian or Alaska Native (65%). A large portion of these patients (20.5%) resulted in GIA calls of EUR, likely due to the admixture of Native American and European populations. However, consensus GIA calls had high specificity for these groups (0.99-1) and high precision for the CAS/SIB group, showing that the workflow errs on the side of less “false-positive” AMR and CAS/SIB ancestry calls. Precision was reduced for the AMR group (0.71) due to some American Indian or Alaska Native patients resulting in GIA calls of AMR, due to inherent relatedness between North and Central/South American-based Native Americans. This also provides an example of how GIA is potentially capturing biological ancestry (i.e. Hispanic or Latino patients with Native American versus European lineages) compared to SIRE, which captures social and cultural constructs (i.e. labeled Hispanic or Latino regardless of lineage but based on where they currently reside, where they or their family immigrated from, etc.).

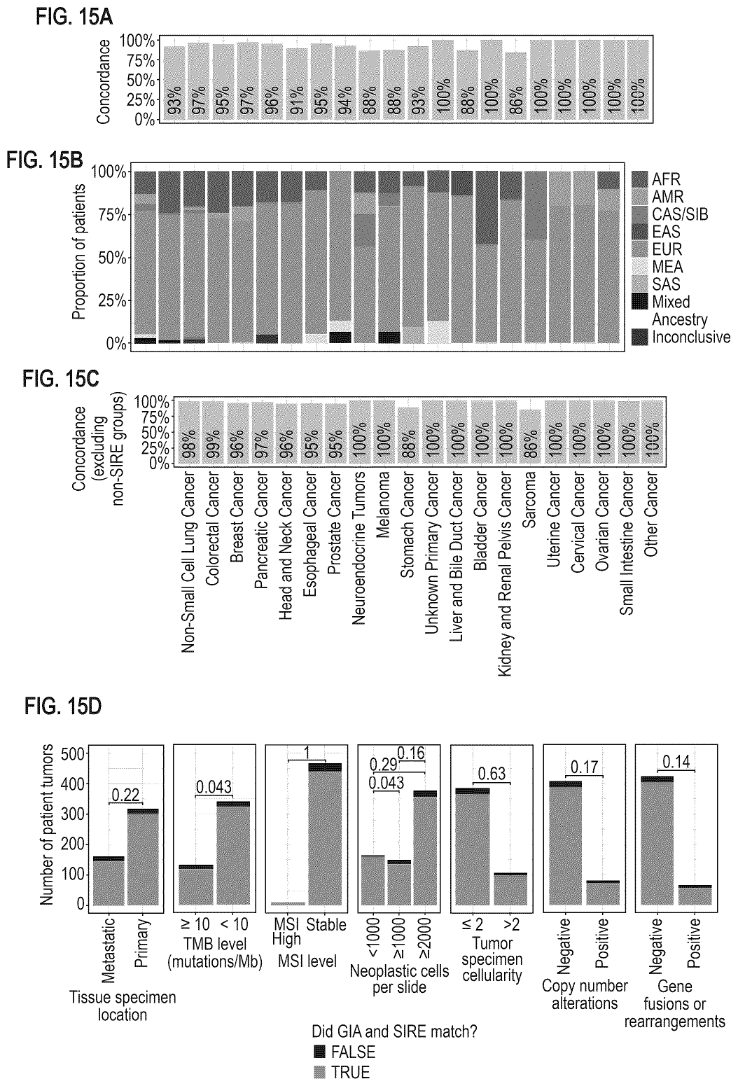

Concordance between consensus GIA calls and SIRE varied by tumor type, with the lowest concordances seen among melanoma (81%), sarcomas (86%), stomach cancer (88%), and bladder cancer (88%) (FIG. 15A). Other tumor types ranged from 91% to 100% concordance between GIA calls and SIRE with an average concordance of 94.7% (FIG. 15A). Lower concordances for melanoma and bladder tumor types were mostly driven by higher proportions of GIA groups not covered by SIRE (i.e. MEA and Mixed Ancestry; FIG. 15B). After removing GIA groups not covered by SIRE, concordances for melanoma and bladder groups increased to 93% and 100%, respectively, and the overall average increased to 97.1% (FIG. 15C).

Concordance between consensus GIA calls and SIRE was not significantly influenced by whether a patient's tumor was derived from a primary or metastatic site (P=22), the number of neo-plastic cells in the tumor specimen (P≥0.1), tumor cellularity (P=48), or genomic characteristics of the tumor including MSI status (P=1), or presence or absence of copy number alterations (P=29) or gene fusions/rearrangements (P=0.08) (FIG. 15D). Cases that had low TMB (<10 mutations/Mb) had a slightly, but significantly, higher concordance between GIA calls and SIRE when compared to high TMB cases (96% versus 91%) (FIG. 15D). This suggests concordances are not biased by where a tumor specimen is derived from, if a tumor exhibits mismatch repair deficiency, or contains larger structural alterations or gene rearrangements; however, the number of somatic mutations in the tumor DNA may slightly influence concordances with SIRE.

### CONCLUSIONS

Using genomic data from 4,274 reference samples from eight geographical populations and 491 tumor samples from patients with SIRE data who underwent CGP testing, a new workflow to obtain accurate GIA was developed and validated. The workflow improves upon previous workflows by expanding the pool of available reference populations, allowing for more comprehensive ancestry inferences, and utilizing consensus-based classification to obtain an accurate and robust GIA call. The GIA workflow had high concordance with patients' SIRE and could expand on SIRE by (i) detecting the ancestry of patients that usually lack appropriate racial categories, (ii) determining what patients have mixed ancestry, and (iii) resolving ancestries of patients in heterogeneous racial categories and who had missing SIRE. The workflow was designed to run on sequencing results from the TSO 500 CGP assay but can readily be extended to other CGP assays, allowing accurate GIA calls to be made across different tests from tumor DNA. Accurate GIA data provide needed information to enable ancestry-aware biomarker research, which can help mitigate cancer outcome disparities, ensure the inclusion of underrepresented groups in clinical research, and help findings be more representative of real-world patient populations with disease enabling targeted therapies and clinical trials to benefit all populations with disease.

### Embodiments

A1. A computer-implemented method comprising:
accessing reference sequencing files and a subject sequencing file, wherein the reference sequencing files and the subject sequencing file are generated as part of performing a next generation sequencing assay;
identifying, using a hybrid variant tool, genomic variants in the reference sequencing files and the subject sequencing file to generate reference variant files and a subject variant file;
generating, by file consolidation using the reference variant files, a datastore formatted file;
performing, by querying the datastore formatted file, joint variant calling to aggregate the variant calls across the reference variant files to generate a final reference variant file;
merging the final reference variant file with the subject variant file to generate a merged variant file;
determining, by principal component (PC) analysis on the merged variant file, reference PCs and subject PCs, wherein the reference PCs and the subject PCs comprise a top set of PCs;
predicting, by a first classification process using the reference PCs and the subject PCs, a first ancestry call based on the correlations found between the reference PCs and subject PCs;
determining, by a second classification process using the reference PCs and the subject PCs, a second ancestry call based on a distance metric of the second classification process, and
determining, by a third classification process using the merged variant file, a third ancestry call based on a maximum likelihood estimation of the third classification process; and
predicting, for the subject sample, a consensus genetically inferred ancestry (GIA) call based on the first ancestry call, the second ancestry call, and the third ancestry call.

A2. The computer-implemented method of embodiment A1, wherein the reference sequencing files comprise individual sequencing files from at least 6 ancestral populations.

A3. The computer-implemented method of embodiment A2, wherein the ancestral populations comprise African, European, Admixed American, East Asian, South Asian, Middle Eastern, Central Asian/Siberian, Oceania populations, or any combination thereof.

A4. The computer-implemented method of embodiment A1, wherein the reference sequencing files are from normal tissue samples.

A5. The computer-implemented method of embodiment A1, wherein the subject sequencing file comprises gene regions corresponding to a comprehensive genome panel.

A6. The computer-implemented of embodiment A5, wherein the comprehensive genome panel is a cancer genome panel, a non-invasive prenatal testing genome panel, or a transplant screening panel.

A7. The computer-implemented method of embodiment A6, wherein the comprehensive genome panel is a cancer genome panel.

A8. The computer-implemented method of embodiment A7, wherein the comprehensive genome panel is the TruSight® Oncology 500 gene panel.

A9. The computer-implemented method of embodiment A5, wherein the comprehensive genome panel is used to filter the reference sequencing files.

A10. The computer-implemented method of embodiment A1, wherein the hybrid variant tool comprises a variant caller integrated into a genomic data analyzer.

A11. The computer-implemented method of embodiment A10, wherein the variant caller uses multi-threading and distributed computing techniques.

A12. The computer-implemented method of embodiment A10, wherein the genomic data analyzer uses FPGA processing.

A13. The computer-implemented method of embodiment A1, wherein the genomic variants comprise single nucleotide polymorphisms (SNPs), insertion/deletions (indels), short tandem repeats (STRs), copy number variants (CNVs), chromosomal rearrangements, or any combination thereof.

A14. The computer-implemented method of embodiment A13, wherein the genomic variants comprise SNPs and indels.

A15. The computer-implemented method of embodiment A1, wherein the top set of PCs comprises at least 20 principal components.

A16. The computer-implemented method of embodiment A1, wherein the first classification method is a correlation-based algorithm that calculates the Pearson correlation between genetic PCs of the subject sample and PCs of every reference sample, extracts the top 1% of calculated correlations, and wherein the first ancestry call is the reference population with the highest number of reference samples represented in the top correlations.

A17. The computer-implemented method of embodiment A16, wherein the correlation-based algorithm outputs more than one ancestry call.

A18. The computer-implemented method of embodiment A1, wherein the second classification method is a k-nearest neighbor algorithm trained on the top set of PCs to predict the second ancestry call, and wherein the second ancestry call is the reference population that appears the most frequently in the k nearest neighbors.

A19. The computer-implemented method of embodiment A1, wherein the third classification method is an admixture method, and wherein the third ancestry call is the reference population with the highest ancestry fraction.

A20. The computer-implemented method of embodiment A1, wherein the predicted consensus GIA call is reported as:
(i) an ancestry type when at least two of either the first, the second, or the third ancestry calls are the same,
(ii) mixed ancestry when all the maximum likelihood estimations from the third classification process are below a threshold, or
(iii) inconclusive when no concordance across the first, second, and third ancestry calls and all the maximum likelihood estimations from the third classification process are below a threshold.

A21. The computer-implemented method of embodiment A20, wherein the threshold is greater than or equal to 0.54.

### Additional Considerations

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine-readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium”, “storage” or “memory” can represent one or more memories for storing data, including read only memory (ROM), random access memory (RAM), magnetic RAM, core memory, magnetic disk storage mediums, optical storage mediums, flash memory devices and/or other machine-readable mediums for storing information. The term “machine-readable medium” includes, but is not limited to portable or fixed storage devices, optical storage devices, wireless channels, and/or various other storage mediums capable of storing that contain or carry instruction(s) and/or data.

While the principles of the disclosure have been described above in connection with specific apparatuses and methods, it is to be clearly understood that this description is made only by way of example and not as limitation on the scope of the disclosure.

What is claimed:

1. A computer-implemented method comprising:
accessing reference sequencing files and a subject sequencing file, wherein the reference sequencing files and the subject sequencing file are generated as part of performing a next generation sequencing assay;
identifying, using a hybrid variant tool, genomic variants in the reference sequencing files and the subject sequencing file to generate reference variant files and a subject variant file;
generating, by file consolidation using the reference variant files, a datastore formatted file;
performing, by querying the datastore formatted file, joint variant calling to aggregate the variant calls across the reference variant files to generate a final reference variant file;
merging the final reference variant file with the subject variant file to generate a merged variant file;
determining, by principal component (PC) analysis on the merged variant file, reference PCs and subject PCs, wherein the reference PCs and the subject PCs comprise a top set of PCs;
predicting, by a first classification process using the reference PCs and the subject PCs, a first ancestry call based on the correlations found between the reference PCs and subject PCs;
determining, by a second classification process using the reference PCs and the subject PCs, a second ancestry call based on a distance metric of the second classification process, and
determining, by a third classification process using the merged variant file, a third ancestry call based on a maximum likelihood estimation of the third classification process; and
predicting, for the subject sample, a consensus genetically inferred ancestry (GIA) call based on the first ancestry call, the second ancestry call, and the third ancestry call.

2. The computer-implemented method of claim 1, wherein the reference sequencing files comprise individual sequencing files from at least 6 ancestral populations, and wherein the ancestral populations comprise African, European, Admixed American, East Asian, South Asian, Middle Eastern, Central Asian/Siberian, Oceania populations, or any combination thereof.

3. The computer-implemented method of claim 1, the subject sequencing file comprises gene regions corresponding to a comprehensive genome panel.

4. The computer-implemented method of claim 1, wherein the hybrid variant tool comprises a variant caller integrated into a genomic data analyzer.

5. The computer-implemented method of claim 4, wherein the variant caller uses multi-threading and distributed computing techniques, and wherein the genomic data analyzer uses FPGA processing.

6. The computer-implemented method of claim 1, wherein the top set of PCs comprises at least 20 principal components.

7. The computer-implemented method of claim 1, wherein the first classification method is a correlation-based algorithm that calculates the Pearson correlation between genetic PCs of the subject sample and PCs of every reference sample, extracts the top 1% of calculated correlations, and wherein the first ancestry call is the reference population with the highest number of reference samples represented in the top correlations.

8. The computer-implemented method of claim 1, wherein the second classification method is a k-nearest neighbor algorithm trained on the top set of PCs to predict the second ancestry call, and wherein the second ancestry call is the reference population that appears the most frequently in the k nearest neighbors.

9. The computer-implemented method of claim 1, wherein third classification method is an admixture method, and wherein the third ancestry call is the reference population with the highest ancestry fraction.

10. The computer-implemented method of claim 1, wherein the predicted consensus GIA call is reported as:
(i) an ancestry type when at least two of either the first, the second, or the third ancestry calls are the same,
(ii) mixed ancestry when all the maximum likelihood estimations from the third classification process are below a threshold, or
(iii) inconclusive when no concordance across the first, second, and third ancestry calls and all the maximum likelihood estimations from the third classification process are below a threshold.

11. A system comprising:
one or more processors; and
one or more computer-readable media storing instructions which, when executed by the one or more processors, cause the system to perform operations comprising:
accessing reference sequencing files and a subject sequencing file, wherein the reference sequencing files and the subject sequencing file are generated as part of performing a next generation sequencing assay;
identifying, using a hybrid variant tool, genomic variants in the reference sequencing files and the subject sequencing file to generate reference variant files and a subject variant file;
generating, by file consolidation using the reference variant files, a datastore formatted file;
performing, by querying the datastore formatted file, joint variant calling to aggregate the variant calls across the reference variant files to generate a final reference variant file;
merging the final reference variant file with the subject variant file to generate a merged variant file;
determining, by principal component (PC) analysis on the merged variant file, reference PCs and subject PCs, wherein the reference PCs and the subject PCs comprise a top set of PCs;
predicting, by a first classification process using the reference PCs and the subject PCs, a first ancestry call based on the correlations found between the reference PCs and subject PCs;
determining, by a second classification process using the reference PCs and the subject PCs, a second ancestry call based on a distance metric of the second classification process, and
determining, by a third classification process using the merged variant file, a third ancestry call based on a maximum likelihood estimation of the third classification process; and
predicting, for the subject sample, a consensus genetically inferred ancestry (GIA) call based on the first ancestry call, the second ancestry call, and the third ancestry call

12. The system of claim 11, wherein the reference sequencing files comprise individual sequencing files from at least 6 ancestral populations, and wherein the ancestral populations comprise African, European, Admixed American, East Asian, South Asian, Middle Eastern, Central Asian/Siberian, Oceania populations, or any combination thereof.

13. The system of claim 11, wherein the subject sequencing file comprises gene regions corresponding to a comprehensive genome panel.

14. The system of claim 11, wherein the hybrid variant tool comprises a variant caller integrated into a genomic data analyzer.

15. The system of claim 14 wherein the variant caller uses multi-threading and distributed computing techniques, and wherein the genomic data analyzer uses FPGA processing.

16. The system of claim 11 wherein the first classification method is a correlation-based algorithm that calculates the Pearson correlation between genetic PCs of the subject sample and PCs of every reference sample, extracts the top 1% of calculated correlations, and wherein the first ancestry call is the reference population with the highest number of reference samples represented in the top correlations.

17. The system of claim 11 wherein the second classification method is a k-nearest neighbor algorithm trained on the top set of PCs to predict the second ancestry call, and wherein the second ancestry call is the reference population that appears the most frequently in the k nearest neighbors.

18. The system of claim 11 wherein third classification method is an admixture method, and wherein the third ancestry call is the reference population with the highest ancestry fraction.

19. The system of claim 11 wherein the predicted consensus GIA call is reported as:
(i) an ancestry type when at least two of either the first, the second, or the third ancestry calls are the same,
(ii) mixed ancestry when all the maximum likelihood estimations from the third classification process are below a threshold, or
(iii) inconclusive when no concordance across the first, second, and third ancestry calls and all the maximum likelihood estimations from the third classification process are below a threshold.

20. One or more non-transitory computer-readable media storing instructions which, when executed by one or more processors, cause a system to perform operations comprising:
accessing reference sequencing files and a subject sequencing file, wherein the reference sequencing files and the subject sequencing file are generated as part of performing a next generation sequencing assay;
identifying, using a hybrid variant tool, genomic variants in the reference sequencing files and the subject sequencing file to generate reference variant files and a subject variant file;
generating, by file consolidation using the reference variant files, a datastore formatted file;
performing, by querying the datastore formatted file, joint variant calling to aggregate the variant calls across the reference variant files to generate a final reference variant file;
merging the final reference variant file with the subject variant file to generate a merged variant file;
determining, by principal component (PC) analysis on the merged variant file, reference PCs and subject PCs, wherein the reference PCs and the subject PCs comprise a top set of PCs;
predicting, by a first classification process using the reference PCs and the subject PCs, a first ancestry call based on the correlations found between the reference PCs and subject PCs;
determining, by a second classification process using the reference PCs and the subject PCs, a second ancestry call based on a distance metric of the second classification process, and
determining, by a third classification process using the merged variant file, a third ancestry call based on a maximum likelihood estimation of the third classification process; and
predicting, for the subject sample, a consensus genetically inferred ancestry (GIA) call based on the first ancestry call, the second ancestry call, and the third ancestry call.
